(19) United States
(12) Patent Application Publication
(10) Pub. No.: US 2024/0041557 A1
(43) Pub. Date: Feb. 8, 2024

(54) APPARATUS, METHOD AND COMPUTER PROGRAM PRODUCT FOR CONTROLLING OPERATIONS OF DEVICES IN A MEDICAL SYSTEM

(71) Applicant: Sony Group Corporation, Tokyo (JP)

(72) Inventor: Junichi YOKOTA, Stuttgart (DE)

(73) Assignee: Sony Group Corporation, Tokyo (JP)

(21) Appl. No.: 18/266,286

(22) PCT Filed: Nov. 18, 2021
(86) PCT No.: PCT/EP2021/082210

§ 371 Date: Jun. 9, 2023

(30) Foreign Application Priority Data
Dec. 16, 2020 (EP) 20214460.6

(51) Int. Cl. A61B 90/30; G16H 40/67; G06F 3/0482

(52) U.S. Cl. CPC A61B 90/30; G16H 40/67; G06F 3/0482

(57) ABSTRACT

An apparatus, method and computer program product for controlling operations of a plurality of devices of a medical system are provided. The medical system comprises a plurality of different types of devices of a plurality of different specifications, wherein devices of a different type and/or devices of the same type having a different specification have a different set of available control operations.

### BACKGROUND

### Field of the Disclosure

The present invention relates to an apparatus, method and computer program for controlling operations of devices in a medical system.

### Description of the Related Art

The “background” description provided herein is for the purpose of generally presenting the context of the disclosure. Work of the presently named inventors, to the extent it is described in the background section, as well as aspects of the description which may not otherwise qualify as prior art at the time of filing, are neither expressly or impliedly admitted as prior art against the present invention.

Modern systems (such as medical systems or the like) are very complex and often comprise a large number of different types of devices. Individual devices within the system often perform a number of different tasks. Some of these tasks may be quite specific to the model and type of device itself. However, the devices in a system usual perform tasks which complement the tasks that are performed by other devices within the system. For instance, imaging devices may perform imaging tasks, while lighting devices may illuminate the scene such that the imaging device can capture a high quality image of the scene.

As the complexity of these systems increases, it can be difficult to ensure that all the individual devices perform the appropriate tasks and operations. Indeed, it can become very difficult for a user to operate the devices of the system with ease and efficiency. These issues are exacerbated as both the size of the system and the range of different types of devices of the system increase.

In some situations, such a medical system or the like, harmonious and reliable control of the devices of the system can be particularly important. That is, in a medical system or the like, difficulties in controlling the devices of the system can interrupt the performance of surgical task. This can have certain implications or negative consequences for the outcome of a surgical task.

It is an aim of the present disclosure to address these issues.

### SUMMARY

In a first aspect of the present disclosure, an apparatus for controlling operations of a plurality of devices of a medical system is provided, the medical system comprising a plurality of different types of devices of a plurality of different specifications, wherein devices of a different type and/or devices of the same type having a different specification have a different set of available control operations, the apparatus comprising circuitry configured to: acquire information regarding the set of control operations which are available for each device of the plurality of devices of the medical system; acquire information regarding configurations which can be chosen for each type of control operation for each device of the medical system; generate a common set of control operations for controlling the plurality of devices of the medical system, the common set of control operations comprising at least one type of control operation which can be performed by each device of the plurality of devices of the medical system; generate a mapping between the common set of control operations and the configuration which can be chosen for each type of control operation forming part of the common set of control operations for each device of the plurality of devices of the medical system; produce a signal to cause an external display device to display the common set of control operations as a single user interface for the control of the plurality of devices of the medical system; and control one or more of the devices of the medical system using a selection of a control operation from the common set of control operations and the mapping which has been generated.

In a second aspect of the disclosure, an apparatus for controlling operations of a plurality of devices of a medical system is provided, the medical system comprising a plurality of different types of devices of a plurality of different specifications, wherein devices of a different type and/or devices of the same type having a different specification have a different set of available control operations, the apparatus comprising circuitry configured to: acquire an activation signal, the activation signal being generated when a user activates a second medical system; and control the plurality of devices of the medical system to cause the plurality of devices to operate in an operation mode in accordance with the second medical system which has been activated by the user; wherein the operation mode for each device of the plurality of devices of the medical system is defined by the available control operations for that device.

In a third aspect of the disclosure, a method of controlling operations of a plurality of devices of a medical system is provided, the medical system comprising a plurality of different types of devices of a plurality of different specifications, wherein devices of a different type and/or devices of the same type having a different specification have a different set of available control operations, the method comprising the steps of: acquiring information regarding the set of control operations which are available for each device of the plurality of devices of the medical system; acquiring information regarding configurations which can be chosen for each type of control operation for each device of the medical system; generating a common set of control operations for controlling the plurality of devices of the medical system, the common set of control operations comprising at least one type of control operation which can be performed by each device of the plurality of devices of the medical system; generating a mapping between the common set of control operations and the configuration which can be chosen for each type of control operation forming part of the common set of control operations for each device of the plurality of devices of the medical system; producing a signal to cause an external display device to display the common set of control operations as a single user interface for the control of the plurality of devices of the medical system; and controlling one or more of the devices of the medical system using a selection of a control operation from the common set of control operations and the mapping which has been generated.

In a fourth aspect of the disclosure, a method of controlling operations of a plurality of devices of a medical system is provided, the medical system comprising a plurality of different types of devices of a plurality of different specifications, wherein devices of a different type and/or devices of the same type having a different specification have a different set of available control operations, the method comprising the steps of: acquiring an activation signal, the activation signal being generated when a user activates a second medical system; and controlling the plurality of devices of the medical system to cause the plurality of devices to operate in an operation mode in accordance with the second medical system which has been activated by the user; wherein the operation mode for each device of the plurality of devices of the medical system is defined by the available control operations for that device.

In a fifth aspect of the disclosure, a computer program product comprising instructions which, when the instructions are implemented by a computer, cause the computer to perform the method of controlling operations of a plurality of devices of a medical system in accordance with the present disclosure is provided.

According to embodiments of the disclosure, a user is able to control multiple different types and models of devices with increased accuracy and control. This improves efficiency when using a plurality of different devices to perform a complex task, for example (such as in a medical system). Of course, the present disclosure is not particularly limited to the above-identified technical effects. Other advantageous technical effects will become apparent to the skilled person when reading the disclosure.

The foregoing paragraphs have been provided by way of general introduction, and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates an example situation to which embodiments of the disclosure may be applied;

FIG. 2 illustrates an example system to which embodiments of the disclosure may be applied;

FIG. 3 illustrates a plurality of different devices in accordance with embodiments of the disclosure;

FIG. 4 illustrates an example set of device specifications in accordance with embodiments of the disclosure;

FIG. 5 illustrates an example configuration of an apparatus in accordance with embodiments of the disclosure;

FIG. 6 illustrates an example set of device specifications in accordance with embodiments of the disclosure;

FIG. 7A illustrates an example control display produced in accordance with embodiments of the disclosure;

FIG. 7B illustrates an example control display produced in accordance with embodiments of the disclosure;

FIG. 7C illustrates an example control display produced in accordance with embodiments of the disclosure;

FIG. 8A illustrates an example control display produced in accordance with embodiments of the disclosure;

FIG. 8B illustrates an example control display produced in accordance with embodiments of the disclosure;

FIG. 8C illustrates an example control display produced in accordance with embodiments of the disclosure;

FIG. 9 illustrates an example set of device configurations in accordance with embodiments of the disclosure;

FIG. 10A illustrates an example control display produced in accordance with embodiments of the disclosure;

FIG. 10B illustrates an example control display produced in accordance with embodiments of the disclosure;

FIG. 10C illustrates an example control display produced in accordance with embodiments of the disclosure;

FIG. 11 illustrates a method of controlling operations of a plurality of devices of a medical system in accordance with embodiments of the disclosure;

FIG. 12 illustrates an example configuration of an apparatus in accordance with embodiments of the disclosure;

FIG. 13A illustrates an example timing chart in accordance with embodiments of the disclosure;

FIG. 13B illustrates an example timing chart in accordance with embodiments of the disclosure;

FIG. 13C illustrates an example timing chart in accordance with embodiments of the disclosure;

FIG. 14 illustrates a method of controlling operations of a plurality of apparatus of a medical system in accordance with embodiments of the disclosure;

FIG. 15 illustrates an example device in accordance with embodiments of the disclosure.

### DESCRIPTION OF THE EMBODIMENTS

Referring now to the drawings, wherein like reference numerals designate identical or corresponding parts throughout the several views.

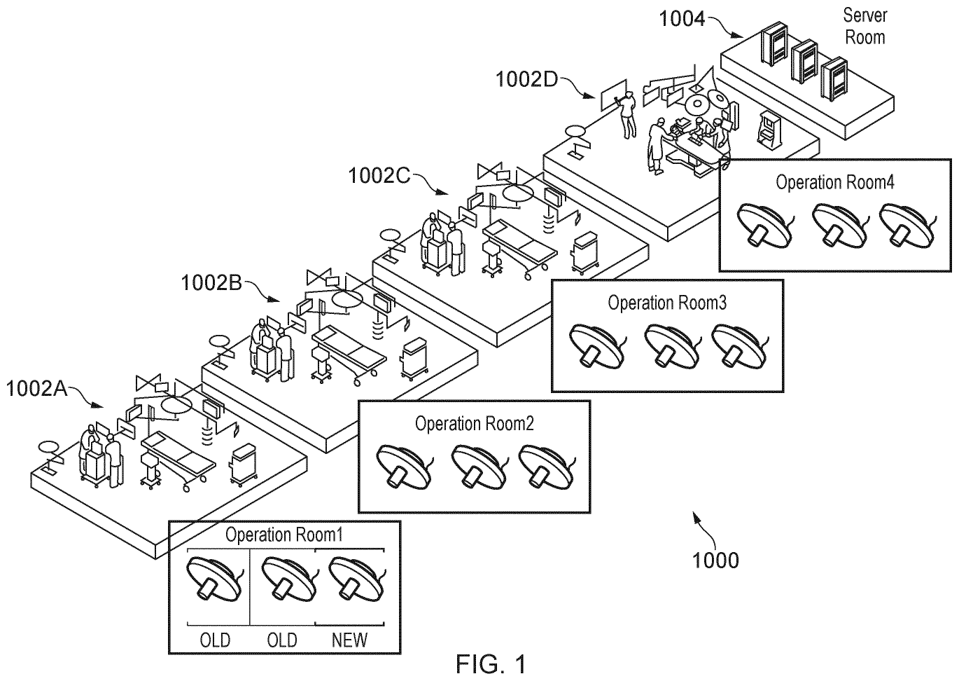

FIG. 1 illustrates an example situation to which embodiments of the disclosure may be applied. Specifically, FIG. 1 shows an example of a system of medical devices as may be present within a medical facility 1000 such as a hospital, dental practice or the like.

A number of individual operation rooms 1002A to 1002D are located within the medical facility 1000. Each of these operation rooms 1002A to 1002D is a room which is equipped with a number of medical devices that are required in order for a surgeon to perform a certain operation or procedure on a patient. For example, each of the rooms 1002A to 1002D shown in the example of FIG. 1 include at least an operation chair and/or table on which a patient can be located for the duration of the operation or medical procedure. Other devices which are included within the operation rooms 1002A to 1002D are described in more detail below.

In recent years, operating rooms (such as operating rooms 1002A to 1002D described above) have become increasingly complex systems, within which each device is inter-connected over an IP network.

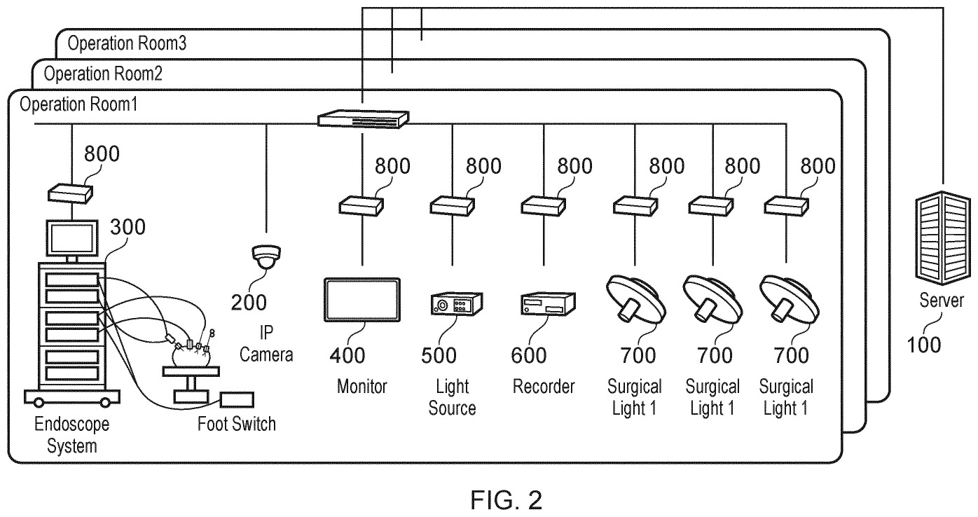

A more detailed example of the configuration of an operating room such as operation rooms 1002A to 1002D is shown in FIG. 2. More generally, FIG. 2 illustrates an example system to which embodiments of the disclosure may be applied.

The surgical system of the example of FIG. 2 (as may be located in the operation rooms 1002A to 1002D of the medical facility 1000 of FIG. 1 of the present disclosure) is connected to a server 100 (which may be located in server room 1004). The surgical system itself includes an operating field camera 200, an endoscope system 300, a monitor 400, a light source 500, a recorder 600, a number of surgical lights 700 and a number of IP converters 800. In the surgical system shown in FIG. 2 of the present disclosure, images, control commands, statuses, and the like are transmitted and received over the IP network in order to control the devices of the medical system during a surgical or medical procedure. For example, images from a camera (such as the IP camera and/or an endoscopic imaging device) are displayed on monitor 400 (an example of an external display device). However, input instructions or commands issued by a user (e.g. using a control displayed on the monitor 400) may be transmitted across the IP network to a device such as light source 500.

Now, it will be appreciated that while an endoscope system 300 is shown in the example of FIG. 2 of the present disclosure, more generally a medical video source configured to output medical video source information of a medical video of a medical procedure performed on a patient in an operating room may be provided. That is, the type of medical video source provided will depend upon the type of surgery which is to be performed.

Furthermore, the operating field camera 200 is, more generally, a video source configured to output video source information. An IP camera and recorder belong to this category and are an example of an operating field camera 200. This provides a visual overview of any activity which occurs within the operation room.

In addition, video feed from the operation room may be obtained from remotely controllable cameras, laparoscopic cameras, or a surgeons head mounted camera.

The IP converter 800 may perform interface conversion between a given device and the IP network when that device does not have an intrinsic IP network interface. The IP converter 800 may also have functions such as image processing in addition to interface conversion. The server 100 shown in FIG. 2 of the present disclosure can control a device directly connected via the IP network, a device connected via the IP converter 800, or the IP converter 800 itself. As such, the IP converter converts the video source information into packetized video data and additional control signals for transmission over the IP network between the medical devices and the server in the server room 1004.

As explained above, the monitor 400 (or other display device) is used in order to display certain information to the surgeon during the surgical or medical procedure. In fact, a number of display devices (such as display screens) may be located within each of the operation rooms 1002A to 1002D. These display devices can be used in order to display certain information to the surgeon, or other medical practitioner, during surgery. This information may include information regarding the patient or information regarding the operation which is to be performed. Moreover, the display devices can be used in order to display images from a number of medical imaging devices (such as an endoscopic imaging device). The type of monitor is not particularly limited, but may include a display device capable of displaying video in 4K resolution, for example.

Furthermore, video from the video capture devices (such as the operating field camera 200) may be recorded and/or archived on server 100 such that it can be retrieved at a later stage. Alternatively, said video may be recorded in or by the recorder 600. Moreover, video from the video capture devices may be provided over the IP network to a remote viewer.

Surgical (or operating) lights 700 are lighting devices which are used during surgical operations or other medical procedures. The surgical lights may be used in order to assist a surgeon during a surgical operation by providing illumination of a local area of the patient. Some surgical lights include ambient lights for use in minimal invasion surgery. In particular, each operation room may have its own lighting system and lighting devices. It is very important during medical procedures (such as a surgical operation) that the level of lighting is very precisely controlled in order to give the surgeon (or other medical practitioner) the best view of the surgical scene. Moreover, the level of light or other lighting requirements may change over the course of the medical procedure. That is, at some stages during the medical procedure a high level of ambient light may be required. However, at other stages (such as when using a sensitive medical imaging device or the like) a low level of ambient light may be required. Owing to differences between the medical devices (such as medical lighting devices) it may be difficult for a surgeon, surgical team or medical practitioner to accurately and reliably adjust the light in the operation room when changing between the operation rooms.

Finally, an operation room controller or control device (optionally located in the server 100 of FIG. 2 of the present disclosure) may be configured to control a network configuration and establish a connection between the source-side IP (internet protocol) converter and the output-side IP converter. In some examples, the operation room controller can control multiple operating rooms (such as operation rooms 1002A to 1002D of FIG. 1 of the present disclosure). That is, even the devices of the different operation rooms may be interconnected over the IP network.

Of course, it will be appreciated that the medical equipment which is located within each of the operation rooms is not particularly limited to the above described equipment. That is, the medical equipment which is located in the operation room may vary depending on the nature of the upcoming operation which is to be performed. The configuration of the operation room may also vary between the individual operation rooms 1002A to 1002D. Operation rooms 1002A to 1002C may be operation rooms which are used for general operations or medical procedures, while operation room 1002D may be a specialist room which is equipped with certain medical equipment specific to a certain type of medical operation or procedure.

Furthermore, the equipment and devices located in the operation rooms may be upgraded over time as new equipment is produced. The new equipment may be connected to the IP network using an IP convertor 800 if the new equipment does not have intrinsic IP network capability.

Returning now to FIG. 1 of the present disclosure, the medical facility 1000 may also include a server room 1004. The server room may host a number of specialised or dedicated servers and/or computing devices which are used in order to provide control and networking facilities to individual client computing devices located in the operation rooms 1002A to 1002D. Specifically, the server room may include a number of computer storage devices which are used in order to store information which can then be accessed by individual client computing devices located in the operation rooms 1002A to 1002D. This information may include information regarding patients and/or information regarding upcoming operations or medical procedures. The individual client computing devices (or terminals) in the operation rooms 1002A to 1002D may be securely connected to the servers in the server room over a network. Examples of this network include both wired and wireless networks. Indeed, the client terminals and computing devices in each operation room may also be connected to the servers of the server room 1004 over an IP network.

A surgeon, surgical team or other medical practitioner may move between operation rooms over time depending on the type of medical operation or procedure which is to be performed. That is, the surgeon or surgical team may be located in a first operation room for a first procedure and a second, different, operation room for a second procedure. Since the type of equipment present in the operation room may vary between operation rooms, the surgeon, surgical team or other medical practitioner may experience certain difficulty in controlling the medical equipment when changing between operation rooms.

For example, if the surgeon becomes accustomed to using a first set of controls in order to control a device (such as the surgical light) in a first operation room, then the surgeon may experience difficulty in controlling a surgical light in a different operation room (if that surgical light has a different set of controls, for example).

However, even when the surgeon, surgical team or medical practitioner is well acquainted with the medical devices which are located in a certain operation room (such as operation room 1002A), certain difficulties may still be experienced by the surgeon when controlling the medical devices (such as the surgical lighting devices). Firstly, there may a large number of lighting devices in the operation room. Owing to the large number of lighting devices it can therefore be difficult for the surgeon, surgical team or medical practitioner to control the lighting devices effectively. Moreover, even within an operation room there may be a number of different types of lighting devices (such as surgical lighting devices being made by different manufacturers). This can further increase the difficulty for the surgeon, surgical team or other medical practitioners when seeking to adjust or control the lighting devices.

Consider the example medical facility shown in FIG. 1 of the present disclosure. Operation room 1004 may comprise surgical lights or lighting devices of a first type (being made by a first manufacture). This is different to the type of surgical lights or lighting devices which are used in each the operation rooms 1002A, 1002B and 1000C respectively. Moreover, in operation room 1002A while the surgical lights or lighting devices may be of the same type (being issued by the same vendor), a number of the surgical lights or lighting devices may be of a first specification while other surgical lights or lighting devices may be of a second different specification. In this regard, a specification of a device (including a specification of a surgical lighting device) describes the external and internal operational characteristics of the device. These may vary between different devices issued by the same vendor—thus further increasing the difficulty when trying to control a plurality of devices of a medical system.

Differences between the devices (such as the surgical lights) in a single operation room may occur where further surgical lights or lighting devices have been added to the operation room over time. Alternatively, this may occur where an individual surgical light or lighting device has been upgraded or replaced since the operation room was initial constructed.

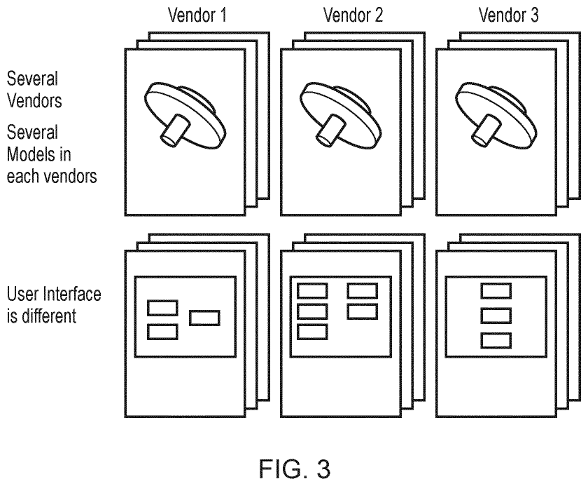

FIG. 3 illustrates a plurality of different devices in accordance with embodiments of the disclosure. In particular, surgical lighting devices from five different manufacturers (or vendors) are shown. These surgical vendors may include vendors such as Merivaara, Draeger, Striker, Storz and Steris, for example. However, the surgical lights which can be used are not particularly limited to these specific examples. Surgical lights from other manufactures (or vendors) may be used as required depending on the situation to which the embodiments of the disclosure are applied. Furthermore, these surgical lights are examples of surgical lights which may be present within each of the operation rooms 1002A, 1002B, 1002C and 1002D of the medical facility 1000. However, it will be appreciated that the present disclosure is not limited to surgical lighting devices obtained from these specific manufacturers (or vendors).

As illustrated in FIG. 3, each of the different manufacturers (or vendors) of surgical lights may also provide several different models of surgical lights (each having different specifications).

Surgical lights such as those illustrated in FIG. 3 of the present disclosure may be controlled in a number of different ways. For example, the lights may be controlled using a user interface displayed on a display device (such as monitor 400) or displayed on another type of control device. The manner by which these surgical lights are natively controlled is not particularly limited in accordance with embodiments of the disclosure.

However, it will be appreciated that, generally, the user interface (or other control mechanism) which is used in order to control the surgical lights such as those illustrated in FIG. 3 of the present disclosure varies amongst different manufacturers. Moreover, each device may have a different set of control operations which are available (control operations being functions which can be set or controlled for each of the devices in order to modify certain attributes or behaviours of the surgical light). Indeed, the control mechanism for each surgical light may also vary between different models of the surgical light produced by the same manufacturer (or vendor).

Therefore, a specification of a device (e.g. a surgical light) describes both the control operations which can be performed for a device and the individual configurations which are available for each of those control operations. More generally, configurations which can be chosen for each type of control operation include at least one of: values, settings, options and/or scales which can be chosen for each type of control operation.

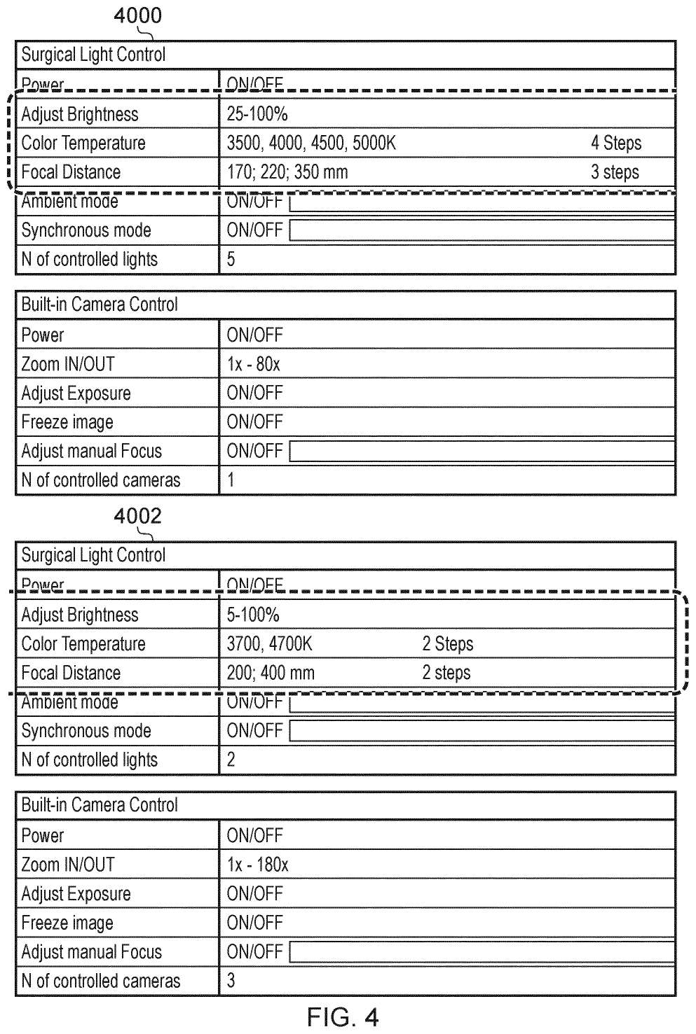

FIG. 4 illustrates an example of device specifications in accordance with embodiments of the disclosure. Two different device specifications 4000 and 4002 for two different surgical lighting devices are shown.

In the example of FIG. 4, the specifications 4000 and 4002 show that the surgical lights have a number of different available control operations (control functions). The control operations of the lighting devices which are described by the specifications include a ‘Focal Distance’ control operation (to control the focal distance of the surgical light), a ‘N of controlled light number’ control operation (to control the number of sub-lights of the lighting device which are activated), a ‘Color Temperature’ control operation (to control the colour temperature of the light), a ‘Adjust Brightness’ control operation (to adjust the brightness of the surgical light) and the like. These control operations can thus be used in order to control the configuration (set of attributes) of surgical lights.

However, it will be appreciated that the control operations of a device (or surgical lighting device) are not limited to the example control operations which are shown in FIG. 4. Other control operations which can be used in order to control the configurations of the surgical lights can be used (such as control operations to control the position and/or orientation of the lights, for example).

The control operations which are described in the specifications 4000 and 4002 are the same in this example. However, this may not necessarily be the case. For example, some surgical lights may have a control operation which enables control of colour temperature while other surgical lights may not.

Nevertheless, even though the control operations of the surgical lights in this example are the same, the configurations which can be selected for each control operation vary between the respective lighting devices. For example, specification 4000 describes that the first lighting device has four different available colour temperatures of 3500, 4000, 4500 and 5000K (i.e. four different colour temperature configurations). In contrast, specification 4002 describes that the second lighting device has only 3700K and 4700K available as colour temperatures (i.e. two different colour temperature configurations).

Differences between device specifications (i.e. differences between control operations (control functions) and/or differences between configurations (settings or values) which can be chosen for each of the available control functions) may be present even amongst lighting devices which are produced by the same manufacturer. For example, a first model of lighting device produced by a first manufacturer may have a different specification than a second model of lighting device produced by the same manufacture.

Differences between the specifications of the devices can lead to variations between the user interfaces (or other control mechanisms) which is used in order to control the surgical lights which have been produced even by the same manufacturer.

In other words, surgical lights or lighting devices which have been supplied by multiple different manufactures (or vendors) and/or which comprise a number of different models of surgical lights or lighting devices produced by the same manufacturer may have a very different (and sometimes inconsistent) set of controls. This causes difficulties for surgeons, surgical teams or other medical practitioners when seeking to control the surgical lights or lighting devices during a surgical operation or medical procedure.

Difficulties or delays in controlling the lighting system during an operation or medical procedure may even impact the safety of the operation or medical procedure itself.

As such, for at least these reasons (in addition to the reasons discussed in the Background) it is desired that an apparatus, method and computer program product are provided which enable efficient and accurate control of a plurality of devices of a medical system.

### First Embodiment

In a first embodiment of the disclosure, an apparatus for controlling operations of a plurality of devices of a medical system is provided.

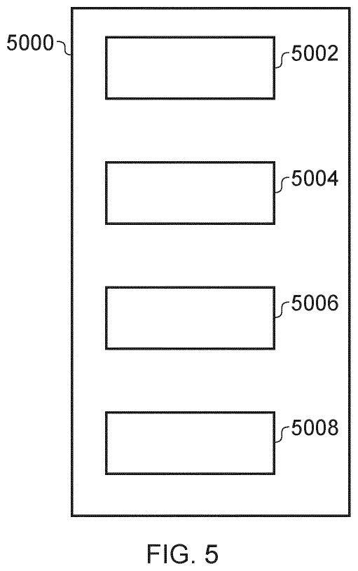

FIG. 5 illustrates an example configuration of an apparatus in accordance with embodiments of the disclosure. The apparatus may be configured for controlling operations of a plurality of devices of a medical system in accordance with embodiments of the disclosure.

The apparatus 5000 comprises an acquiring unit 5002, a generating unit 5004, a producing unit 5006 and a controlling unit 5008.

The acquiring unit 5002 is configured to acquire information regarding the set of control operations which are available for each device of the plurality of devices of the medical system.

Furthermore, the acquiring unit 5002 is configured to acquire information regarding configurations which can be chosen for each type of control operation for each device of the medical system.

Generating unit 5004 is configured to generate a common set of control operations for controlling the plurality of devices of the medical system, the common set of control operations comprising at least one type of control operation which can be performed by each device of the plurality of devices of the medical system. Moreover, generating unit 5004 is configured to generate a mapping between the common set of control operations and the configuration which can be chosen for each type of control operation forming part of the common set of control operations for each device of the plurality of devices of the medical system.

Producing unit 5006 is configured to produce a signal to cause an external display device to display the common set of control operations as a single user interface for the control of the plurality of devices of the medical system.

Furthermore, the controlling unit 5008 of apparatus 5000 is configured to control one or more of the devices of the medical system using a selection of a control operation from the common set of control operations and the mapping which has been generated.

In this manner, the apparatus 5000 enables control of multiple different types and models of devices with increased accuracy and efficiency.

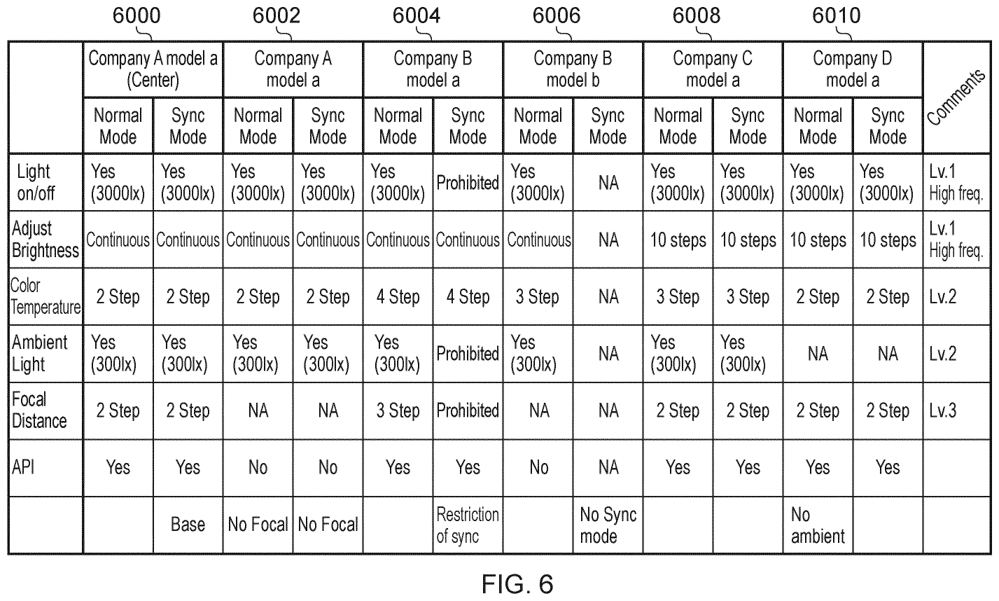

Further details of the apparatus for controlling operations of a plurality of devices of a medical system as described with reference to FIG. 5 of the present disclosure will now be discussed with reference to FIGS. 6 to 10 of the present disclosure.

### <Acquiring Unit>

As described with reference to FIG. 5 of the present disclosure, the acquiring unit 5002 of apparatus 5000 is configured to acquire information regarding the available control operations—and the available configurations of those control operations—for each of the devices of the medical system.

The acquiring unit 5002 may acquire this information from the devices of the medical system in a number of different ways. In some examples, the acquiring unit 5002 may acquire this information directly from the individual devices which are included in the medical system. For example, the acquiring unit 5002 may be configured to communicate with the individual devices using any suitable wired or wireless communication in order to obtain this information. In fact, in some examples the acquiring unit 5002 may communicate with the individual devices over an IP network such as that described with reference to FIG. 2 of the present disclosure in order to obtain this information. However, in other examples, information regarding the available control operations and the available configurations of those control operations for each device may be stored in a memory or other storage unit. The acquiring unit 5002 may acquire the information then from this memory or storage unit over a wired or wireless connection. The memory or other storage unit may be either internal or external to apparatus 5000 itself (and may be part of the server 100 in server room 1004).

FIG. 6 of the present disclosure illustrates an example set of device specifications in accordance with embodiments of the disclosure. These device specifications 6000 to 6010 detail, for each individual device of a medical system, the control operations which are available for the device and the configurations which can be chosen (or set) for each of those control operations.

The example set of device specifications of FIG. 6 is illustrated as a table. However, the information may alternatively be stored in any suitable data structure which enables the relevant information for each device to be acquired by acquiring unit 5002. The device specifications as illustrated in FIG. 6 of the present disclosure are an example of the data from which the acquiring unit can acquire the necessary information regarding the control functions and configurations available for each device of the medical system.

In the example of FIG. 6, there are specifications for six different devices from four different manufacturers (or vendors). These may be the different devices which form part of the medical system described with reference to FIG. 1 of the disclosure (being all those surgical lights which form part of the medical facility 1000).

Examples of control operations available for these devices are: Light on/off, Adjust Brightness, Colour Temperature, Ambient Light on/off, and Focal Distance. However, as shown in specifications 6000 to 6010, not all of these control operations are available for all of the devices. That is, the control operation Ambient Light on/off is not available for the lighting device produced by Company D, for example. This is detailed in specification 6010 (being the specification for the device produced by Company D). The Ambient light control operation is used to produce a comparatively dark light such as may be required during endoscopy surgery, for example. The device produced by Company D does not have this control operation in this example.

Furthermore, some of the devices have different modes of operation (Normal Mode/Sync Mode). Normal mode is a mode where user can control the surgical light (such as the light produced by Company A) individually (i.e. without controlling the other surgical lights in the medical system). On the contrary Sync mode enables control of multiple surgical lights simultaneously. Some of the control operations or configurations which are available for a given device vary even depending on which mode (Normal Mode or Sync Mode) the device is being operated in. For example, the focal distance of the surgical light by Company B may be adjusted using a Focal Distance control operation with 3 different configurations when that device is operated in Normal Mode. However, the focal distance of the device may not be adjusted when the surgical light by Company B is operated in Sync Mode. A company may prohibit certain control operations and/or certain configurations for those control operations in Sync mode in order to prevent the sudden environment change for safety reasons.

As can be seen in the example of FIG. 6, the specifications for the different devices may be similar (including a number of the same control operations). Nevertheless, there may be differences between the control operations and/or configurations available across the devices of the medical system.

Once the information has been acquired by apparatus 1000, the different control operations and the different configurations for those control operations can be passed to the generating unit 5004 of apparatus 1000.

### <Generating Unit 5004>

As described with reference to FIG. 5 of the present disclosure, generating unit 5004 of apparatus 5000 is configured to generate a common set of control operations for controlling the plurality of devices of the medical system, the common set of control operations comprising at least one type of control operation which can be performed by each of the individual devices of the plurality of devices of the medical system.

That is, the generating unit 5004 is configured to look across the different control operations which are available for the different devices which form part of the medical system and identify those control operations which are common to all of the devices. For example, if all of the devices can perform a control operation related to adjustment of focal distance (i.e. the ‘Focal Distance’ control operation of FIG. 6 of the present disclosure), then focal distance will be identified as a control operation which forms part of the common set of control operations for those devices. However, if one or more of the devices cannot perform control operations related to adjustment of focal distance then focal distance will not be identified as a control operation which is common to the devices of the medical system.

As can be seen in FIG. 6 of the present disclosure, there may be a number of control operations which are common across the individual devices (being control operations which are available for those individual devices when operating in at least one of the Normal Mode and/or the Sync Mode). An example of a common control operation in FIG. 6 is the Light on/off control operation, as this control operation is available for each of the individual devices in at least one operation mode (even though its use is restricted in the Sync Mode for each of Company B model a and Company B model b). The Light on/off control operation would therefore form part of the set of common control operations for these devices. Likewise, the control operation Adjust Brightness (for brightness adjustment of the surgical lights) would form part of the common control operations for these devices.

However, the control operation Focal Distance (for adjustment of the focal distance of the surgical light) is not a control operation which forms part of the set of common control operations for these devices because it is not a control operation which is available for Company A model a. Therefore, it cannot be performed by all the devices of the medical system in at least one mode of operation.

It will be appreciated that the manner by which the generating unit 5004 generates the common set of control operations will depend, at least in part, on the format and data structure of the specifications which have been acquired for each of the devices. Nevertheless, the generating unit 5004 uses the information of the control operations which are available for each of the individual devices of the medical system (as acquired by the acquiring unit 5002) in order to identify the common set of control operations for those devices.

Of course, the common set of control operations may be updated or modified as new devices are added to the medical system and/or as old devices are removed from the medical system. As such, the generating unit may be configured to periodically or continuously update the common set of control operations for devices of the medical system.

In addition, generating unit 5004 is configured to generate a mapping between the common set of control operations and the configuration which can be chosen for each type of control operation forming part of the common set of control operations for each device of the plurality of devices of the medical system.

A mapping between the common set of control operations and the individual devices is generated by generating unit 5004 for the reason that even though all the devices of the medical system can perform the control operations of the common set of control operations, different configurations (i.e. settings) may be available for individual devices of the medical system.

Consider, again, the example of FIG. 6 of the present disclosure. Even though the control operation Adjust Brightness (for brightness adjustment) is available for all of the devices of the medical system, the configuration of that control operation varies amongst the individual devices. For example, Company A model a (Center) enables a 2 step adjustment of the Colour Temperature of the surgical light. However, Company B model a enables a 4 step adjustment of the Colour Temperature. Therefore, the configurations which are available for the common set of control operations may vary amongst the devices of the medical system.

The mapping information which is generated by generating unit 5004 in this example would describe the fact that there is a 2 step adjustment of the Colour Temperature of the surgical light available for the surgical light produced by Company A and a 4 step adjustment of the Colour Temperature for the surgical light of Company B. As such, the mapping which is generated by generating unit 5004 details for each of the control operations of the common set of control operations the configurations of that control operation which are available for each of the devices.

The form and structure of the data produced by the generating unit is not particularly limited and will vary in accordance with the situation to which the embodiments of the disclosure are applied. However, the data may, in some examples, be produced in the form of a look up table or the like which describes both the common set of control operations and the configurations which are available for those control operations for each of the devices of the medical system.

In this manner, the information generated by the generating unit 5004 describes both the common set of control operations and the configurations which are available for the common set of the control operations for each of the devices in the medical system. Once generated, this information is passed to the producing unit 5006 of apparatus 5000.

### <Producing Unit>

Once the generating unit 5004 has generated the common set of control operations and the associated mapping with the specifications of the individual devices, the producing unit 5006 of apparatus 5000 is configured to produce a signal to cause an external display device to display the common set of control operations as a single user interface for the control of the plurality of devices of the medical system. In this manner, the producing unit 5006 of apparatus 1000 produces a unique (single) user interface which can be used in order to control the plurality of the devices regardless of the difference of specifications and/or different control operations of those devices.

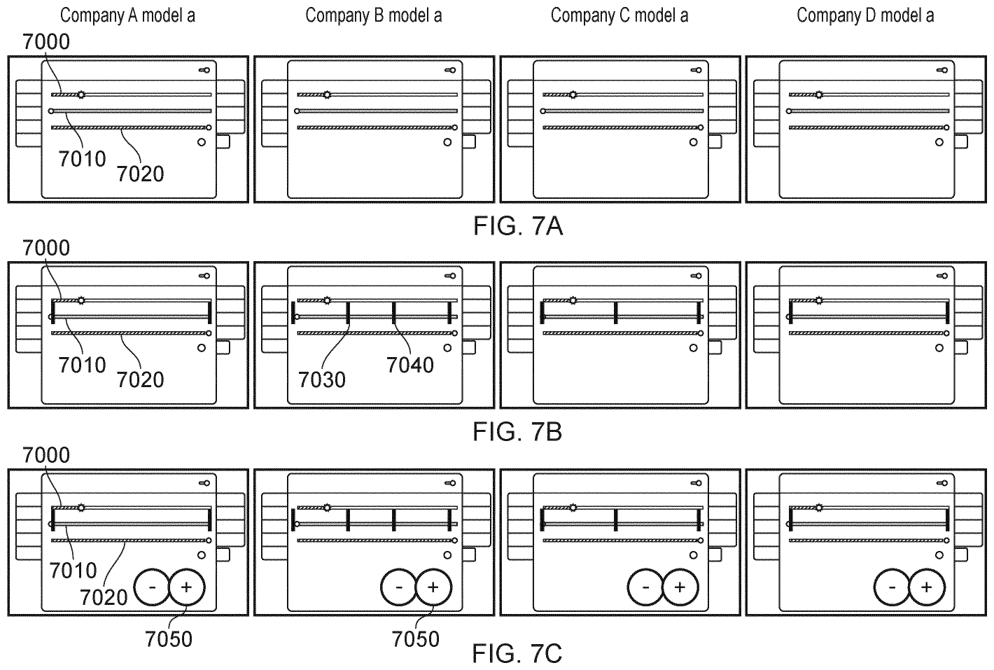

FIG. 7A illustrates an example control display produced in accordance with embodiments of the disclosure. More specifically, FIG. 7A shows an example of a display which is displayed using the display signal produced by the producing unit 5006 of apparatus 1000 in accordance with embodiments of the disclosure. The control display of FIG. 7A may be displayed to a surgeon (or other user) using a external display device such as monitor 400 as described with reference to FIG. 2 of the present disclosure.

In this example, the display is an example of a user interface which is produced for a medical system comprising four different devices (Company A model a, Company B model a, Company C model a, and Company B model a). In this example, the common set of control operations which has been generated by generating unit 5004 comprises control operations 7000, 7010 and 7020. These are the operations which can be performed by all of the devices of the medical system. There may be other control operations which can be performed by one or more of the devices of the medical system. However, since these control operations cannot be performed by all of the devices of the medical system they are not displayed on the unique user interface which is created for the plurality of devices of the medical system.

The four different devices may be the different lighting devices which are found in operation rooms 1002A, 1002B, 1002C and 1002D of the medical facility 1000 as described with reference to FIG. 1 of the present disclosure, for example. However, the present disclosure is not particularly limited in this regard.

The common set of control operations may be an operation to adjust the turn the surgical light on/off 7000, an operation to adjust the colour temperature of the surgical light 7010, and an operation to adjust the brightness of the surgical light 7020. However, the common set of control operations is not particularly limited to these example control operations and will depend on the devices forming the medical system.

As can be seen in FIG. 7A of the present disclosure, the control display (user interface) which is produced for the surgical light ‘Company A model a’ (i.e. the first panel of FIG. 7A) is the same as the control display (user interface) which is produced for the other devices in the medical system (i.e. ‘Company B model a’, ‘Company C model a’, ‘Company D model a’)—that is, the second third and fourth panel of FIG. 7A. Therefore, a device can be controlled by a user with a single type of control display (i.e. user interface) regardless of the type of device which is being controlled. Indeed, in the example of the medical facility 1000 as described with reference to FIG. 1 of the present disclosure, a surgeon can control devices (such as the surgical lights) of the medical system 1000 with increased efficiency and accuracy regardless of the operation room in which the surgeon is located (since the surgeon can use the same control display to control the device regardless of the room in which the surgeon is located).

In some examples, the four panels of the user interface illustrated in FIG. 7A of the present disclosure may be displayed on a single screen (e.g. monitor 400 of FIG. 2 of the present disclosure). In this manner, the user can easily control any of the devices of the medical system by interacting with the corresponding panel on the user interface. In other examples, a limited number of the panels may be displayed. Indeed, in some examples, only a single panel of the user interface of FIG. 7A may be shown on the display screen at any given time. The user can then switch between the panels which are shown on the user interface depending on which device the user wishes to control (i.e. one of ‘Company A model a’, ‘Company B model a’, ‘Company C model a’ and ‘Company D model a’ in this example).

Furthermore, in other examples, only the panels related to a device or set of devices present in the current operation room may be shown on the display screen (even if other devices located in other operation rooms form part of the medical system). That is, if the user is located in operation room 1002B, only the panels related to devices in operation room 1002B will be shown. Then, if the user moves to operation room 1002C, only the panels related to devices in operation room 1002C will be shown to the user. However, since the user interface is common to all the devices, the user can readily understand how to operate the devices in the desired manner even when switching between devices.

The example illustrated in FIG. 7A of the present disclosure relates to operation of the devices in normal mode of operation (where the individual devices of the medical system can be controlled individually by the user).

Accordingly, in the example of FIG. 7A of the present disclosure, a surgeon can manipulate and control the individual devices by interacting with an interactive element on the control display. For example, if the surgeon wishes to adjust the brightness of the surgical light ‘Company A model a’ in operation room 1002A then the surgeon can manipulate the interactive element for the control operation related to brightness adjustment 7020 for ‘Company A model a’. However, if the surgeon moved to operation room 1002B (and operation room 1002B had lights of ‘Company B model a’), the surgeon could adjust the brightness of the surgical lights in operation room 1002B by manipulating the interactive element 1002B for ‘Company B model a’. This reduces technical difficulties in controlling different devices of a medical system since the operation to control the devices is the same regardless of technical differences between the devices.

The onscreen interactive element may be an element such as a button, scroll bar, dial, icon or the like—for example. The manner in which the onscreen interactive element is manipulated by the user (such as a surgeon) is not particularly limited. In some examples the user may interact with the onscreen element using an input device (such as a computer mouse, gaze tracking device or the like). In some examples, the user may interact with the onscreen element using a touch panel, touch screen or the like. Furthermore, in some examples, the user may interact with the onscreen element using an audio control system (e.g. by speaking certain control words or the like). The present disclosure is not particularly limited in this regard.

Since the control display (user interface) which is produced by the producing unit 5006 of apparatus 5000 comprises only those control operations which are common across the devices of the medical system, the surgeon (or any other user) knows that all the control operations which are displayed can be performed regardless of the device which is being controlled. That is, instances where a user attempts to control a device in a manner in which that device cannot be controlled are avoided. In other words, even though the same control display is shown for all of the devices, all of the control operations which are shown can be used with all of the devices of the medical system. As such, user frustration which may occur when a user attempts to initiate a control operation which cannot be performed by a given device is reduced. Moreover, disruption owing to the surgeon (or other user) trying to control the device in a manner which is not compatible with that device is also avoided.

As explained with reference to FIG. 6 of the present disclosure, different devices may have different available configurations with respect to the set of common control operations. That is, even though each of the devices can perform all of the individual control operations 7000, 7010 and 7020, the configurations (settings) which are available for each of the control operations may vary amongst the devices of the medical system.

Accordingly, the mapping which has been generated by generating unit 5006 may be used in order to adjust the control operations for each of the devices (e.g. by applying a scaling to the control operations for each device). This ensures that the user interface can be kept as uniform as possible across the different devices whilst accounting for the different configurations of the control operations.

For example, if the brightness of the surgical light produced by Company A model a can be adjusted between a range which is wider than the range of which the brightness of Company B model a can be adjusted, then the control element 7020 may be scaled for at least one of Company A model a and Company B model a such that the control element 7020 appears the same for the different devices (even though the values which can be controlled or set using the control element 7020 may be different).

As such, even though the available configurations for the common set of control operations may be different amongst the devices of the medical system, the user interface which is produced by the producing unit 5006 of apparatus 1000 is the same.

FIG. 7B illustrates an example control display produced in accordance with embodiments of the disclosure. In this example, additional on screen elements (such as 7030 and 7040) are produced in order to provide individual scaling for the common set of control operations amongst the different devices.
That is, in this example, the onscreen control element 7010 related to the control operation for control of the colour temperature of the lights has a different available configuration for each of the devices of the medical system (i.e. each of ‘Company A model a’, ‘Company B model a’, ‘Company C model a’ and ‘Company D model a’). This information regarding the different configurations for the devices is indicated to the producing unit 5006 which has been generated by the generating unit 5004.

In this example, the surgical light ‘Company A model a’ (as may be present within the operation room 1002A of medical facility 1000) has only two different configurations available for the colour temperature. Therefore, the control display (user interface) produced by producing unit 5006 of apparatus 1000 produces two additional onscreen interactive elements for this control operation of ‘Company A model a’. The surgeon (or other user) may then select (or otherwise interact) with these additional onscreen interactive elements in order to control the colour temperature for the surgical light ‘Company A model a’. Indeed, since the additional onscreen elements are shown the user may immediately understand which configurations of colour temperature are available for the surgical light of ‘Company A model a’. This improves the accuracy and efficiency of operation when the surgeon controls the colour temperature of the surgical lights of ‘Company A model a’.

In contrast, the surgical light ‘Company B model a’ may have four different configurations available for the colour temperature. Therefore, the control display produced by producing unit 5006 of apparatus 1000 produces four additional onscreen interactive elements (including elements 7030 and 7040) for this control operation 7010 of ‘Company B model a’. Each of the additional onscreen interactive elements corresponds to a configuration which is available for that control operation for that device. The user may then select (or otherwise interact) with these additional onscreen interactive elements in order to control the colour temperature for the surgical light of ‘Company B model a’. Indeed, since the additional onscreen elements are shown the user may immediately understand which configurations of colour temperature are available for the surgical light of ‘Company B model a’.

Therefore, even when controlling a number of different devices with a number of different available configurations for the common set of control operations the user can easily and efficiently control the individual devices of the medical system.

FIG. 7C illustrates an example control display produced in accordance with embodiments of the disclosure.

The example illustrated in FIG. 7C of the present disclosure is the same as that illustrated in, and described with reference to, FIG. 7B of the present disclosure. However, in contrast to the example described with reference to FIG. 7B of the present disclosure, an additional onscreen element 7050 is provided in the example of FIG. 7C. This onscreen element may be used in order to step through the available configurations for a selected control operation of a given device.

For example, if there are two available configurations for the colour temperature control operation for the surgical light of ‘Company A model a’, then activating the onscreen element 7050 (when the colour temperature control operation has been selected) will cause the colour temperature to incrementally step (or switch) between these two available configurations. However, for ‘Company B model a’, there are (as described above) four available configurations. Activating the onscreen element 7050 for ‘Company B model a’ will, when the colour temperature control operation has been selected, cause the colour temperature to incrementally step (or switch) between the four available configurations for that device.

As such, even when different configurations are available for the common set of control operations amongst the different devices, those devices can be controlled with improved ease and efficiency through use of a single (unique) control display for the different devices.

As described above, the user interface which is displayed using the display signal produced by the producing unit 5006 of apparatus 1000 contains only those control operations which are common across all of the devices of the medical system. This ensures that there is uniformity of control across all of the devices of the medical system. Nevertheless, there may be certain control operations for certain devices of the medical system which can be performed which cannot be accessed from the common set of control operations. Accordingly, in some examples, if the user wishes to access the control operations specific to the device (and which do not form part of the common set of control operations) then the user may activate a certain onscreen element which enables the additional control operations for that device to be displayed. This may be the native control display (user interface) for that device. This has the advantage that a single user interface can be used in order to control those functions which are common across the plurality of devices of the medical system, while an individual user interface tailored to the individual device can be used in order to access those control operations specific to that device. As such, ease and efficiency of the devices of the medical system is further improved.

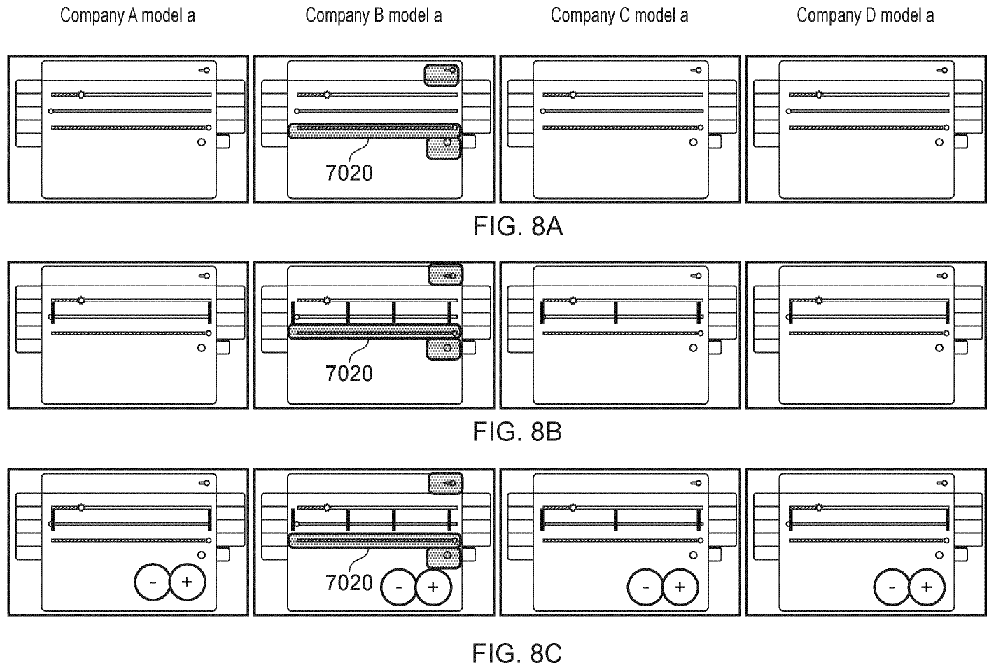

While FIGS. 7A to 7C of the present disclosure describe the control display which is produced for a set of devices operating in the Normal mode (being the mode in which the devices can be controlled individually), a different example situation will be described with reference to FIGS. 8A to 8C of the present disclosure—being a situation where the devices operate in the Sync mode (an operation mode in which different devices can be controlled together in synchronisation through a single user input).

In terms of the Sync Mode, certain safety requirements for operation of the devices should always be respected. That is, while it may be advantageous that certain functions of the devices can be controlled in synchronisation, it may be that certain control operations should be restricted or otherwise prohibited when operating in Sync mode. This may be in order to avoid accidental operation of the devices. For example, ‘Company B model a’ may prohibit ‘Light on/off’, ‘Ambient Light on/off’, and ‘Focal Distance’ functions in sync mode in order to prevent the sudden environment change for safety reasons (this can be seen from the example of FIG. 6 of the present disclosure, for example).

Furthermore, certain configurations and/or control operations may be prohibited from being activated for a certain device in one or more situations in addition to the example situation of the Sync mode. That is, other situations (such as the stage of a medical operation or the like) may also require that certain control operations and/or configurations are prohibited from being selected or otherwise selected by the user.

The user interface illustrated in FIGS. 8A to 8C of the present disclosure is similar to the user interface as illustrated in FIGS. 7A to 7C of the present disclosure. However, in these example illustrations of the control display produced by the producing unit 5006, control operations which are unavailable in Sync mode are visually indicated as prohibited or otherwise excluded from operation. Indeed, in the specific example of FIGS. 8A to 8C the control operations which are unavailable in Sync mode (being, in this specific example, the control operation 7020 for brightness adjustment for ‘Company B model a’) are greyed out in the user interface. These interactive elements cannot be selected by the user such that the user cannot use these control operations to control the surgical lighting device of ‘Company B model a’ while in Sync mode. This ensures that safety of the medical system is maintained even when using a single (unique) control display to control the plurality of devices of the medical system. Moreover, since the user can see that the options are unavailable in Sync mode, the user can understand that those options may be available if the user was to switch to controlling the devices in Normal mode (being where the devices are individually controlled by the user).

In other words, in this example, operating the interactive element 7020 operates for ‘Company A model a’ in sync mode will adjust the brightness configuration for all the surgical lights of the medical system in synchronisation except those lights of ‘Company B model a’. In some examples, only those lights within the operation room in which the surgeon (or other user) is located will be adjusted in synchronization (i.e. a subsection of the devices of the medical system). The surgical lights located in a different operation room would remain unchanged.

More generally, restricting a control operation includes any suitable method of limiting the configurations which can be chosen for that control operation for a certain device in a certain situation or mode or operation.

Of course, the control display produced (user interface) by the producing unit 5006 of the present disclosure are not particularly limited to the specific examples illustrated in FIGS. 7A to 7C and 8A to 8C of the present disclosure. These examples are provided to illustrate certain examples of the control display which can be produced by the producing unit 5006 of apparatus 1000. Importantly, however, according to embodiments of the disclosure, the producing unit 5006 is configured to display the common set of control operations as a single user interface for the control of the plurality of devices of the medical system.

### <Controlling Unit>

As described with reference to FIG. 5 of the present disclosure, the controlling unit 5008 of apparatus 5000 is configured to is configured to control one or more of the devices of the medical system using a selection of a control operation from the common set of control operations and the mapping which has been generated. The selection of the control operation may be received from the user in the form of user input or interaction with the user interface produced by the producing unit 5006 of apparatus 5000.

As described with reference to FIG. 5 of the present disclosure, the producing unit 5006 produces a display signal which instructs an external display device to display a user interface from which a user can control the plurality of devices of the medical system. Specific examples of the user interface have been described with reference to FIGS. 7A to 7C and 8A to 8C of the present disclosure. In particular, it has been described that a number of interactive elements are provided as part of the user interface from which the user can select options which control the devices of medical system. The controlling unit 5008 of apparatus 5000 receives a selection of a control operation which has been made by a user (through use of an input device or the like as described above). Then, the controlling unit 5008 interprets the input which has been received and issues an instruction to the corresponding device (or devices) of the medical system in order that the instruction is carried out by those devices.

Consider a situation where a user is presented with a user interface such as that illustrated in FIG. 7A of the present disclosure. In this example, the four lighting devices of the medical system are located in a first operation room (such as operation room 1002A of FIG. 1 of the present disclosure). During surgery, it is desired that the brightness of the surgical light of ‘Company A model a’ (operating in a first, higher brightness configuration) is decreased. Accordingly, the surgeon (or any other user) can use the user interface which is common across all the devices in order to reduce the brightness of the surgical light of ‘Company A model a’. In particular, the surgeon can interact with the user interface (with any suitable input device such as a touch screen or the like) in order to adjust the configuration of the brightness control operation 7020 for the lighting device of ‘Company A model a’.

In a specific example, the surgeon may interact with a scroll bar such as that illustrated in FIG. 7A in order to reduce the brightness of the surgical light of ‘Company A model a’ to the desired level.

Then, upon receiving the input from the surgeon, the controlling unit 5008 of apparatus 5000 performs a comparison with the mapping information which has been generated by generating unit 5004 to determine the desired configuration of the surgical light. That is, the controlling unit 5008 determines which of the available configurations for the brightness of the surgical light for the light of ‘Company A model a’ corresponds to the input instruction which has been received. In this example, the surgical light has two potential configurations of brightness. As such, upon receiving an input to reduce the brightness of the surgical light of ‘Company A model a’, the controlling unit 5008 identifies that the configuration of the brightness control operation should be changed from the first (higher brightness) configuration to a second (lower brightness) configuration.

In other words, the controlling unit 5008 uses the mapping information in order to determine the input instruction which is received from the user.

Then, once the desired configuration has been determined by the controlling unit 5008 from the input instruction received from the user and the mapping which has been generated by generating unit 5004, the controlling unit 5008 issues an instruction to the surgical light of ‘Company A model a’ in order to change the configuration of the light and thus implement the change in the brightness level.

It will be appreciated that the form of the instruction which is issued by the controlling unit 5008 is not particularly limited. That is, the controlling unit 5008 may issue an instruction in a different data format or the like depending on the device which is being controlled. Indeed, the controlling unit 5008 may have access to a data file with instructions regarding the format of the control instruction which is to be issued to the device. This data file may be stored in a memory either internal or external to apparatus 5000.

In some examples, the controlling unit 5008 may issue the instruction to the device to be controlled over a network such as the IP network described with reference to FIG. 2 of the present disclosure, for example. However, any suitable wired or wireless communication can be used in order to issue the instruction to the device to be controlled as required.

Once the controlling unit 5008 issues the instruction to the device, the device will change its configuration for the corresponding control operation in accordance with the instruction which has been received from the user. Accordingly, the user may operate the plurality of devices of the medical system with improved efficiency using a single user interface which is common across the plurality of devices of the medical system.

### <Additional Modifications>

As described with reference to FIG. 5 of the present disclosure, the generating unit 5004 of apparatus 5000 may generate the common set of control operations based on those control operations which are available for each of the devices in the medical system. However, in some examples, the number of common control operations may be very large. In these situations the user may then be presented with a large number of control operations on the universal user interface which is produced by producing unit 5006. When the number of control operations is large it can, in some situations, become more difficult for the user to reliably control the devices of the medical system.

Accordingly, in some examples, apparatus 5000 may be further configured to categorize the one or more control operations of the common set of control operations in accordance with a frequency of selection.

The frequency of selection (being how often the control operation is used) may be determined in a number of ways. In some examples, the frequency of selection may be a frequency of selection based on the selections which have been made across the medical facility (global frequency). In some other examples, the frequency of selection may be specific to a given user such that the common set of control operations are categorized in accordance with the control operations which are most frequently selected by that specific user. In examples, this may be implemented through use of a user profile or the like.

In examples, the control operations of the common set of control operations having a frequency of selection above a first predetermined threshold value are categorized as a first group of control operations; and the apparatus 5000 is further configured to produce a display signal to cause the external display device (e.g. monitor 400) to display the first group of control operations as a single user interface for the control of the plurality of devices of the medical system.

In this manner, only the common control operations which are most frequently selected by the user are provided to the user on the common user interface. This reduces the number of control operations which are presented to the user on the common (unique) user interface and thus further reduces difficulty of operation of the devices of the medical system when the number of common control operations is large.

Furthermore, apparatus 5000 may also be configured to produce a display signal to cause the external display device to display the first group of control operations and a second group of control operations as a single user interface for the control of the plurality of devices of the medical system in accordance with a selection of a user; wherein the second group of control operations are control operations of the common set of control operations having a frequency of selection below the first predetermined threshold value and above a second predetermined threshold value.

That is, in some situations the user may desire to perform control of a certain operation of the devices using a control operation which is not often used by that user (and which does not, therefore, feature in the most frequently used common control operations). Accordingly, in these situations, the user may instruct the apparatus 5000 to display a second set of control operations (being those control operations which are used less frequently than the first set of control operations, but still more frequently than a number of other control operations which are very rarely used by the user). When the user issues this instruction, the apparatus 5000 may then display a second group of control operations to the user. The user may then select a control operation from the second group of control operations in order to control the desired device. If, however, the common control operation which the user requires is still not listed (featuring in neither of the first or second set of most frequently used control operations) the user may then issue an instruction that all of the common control operations are shown (regardless of their frequency of operation).

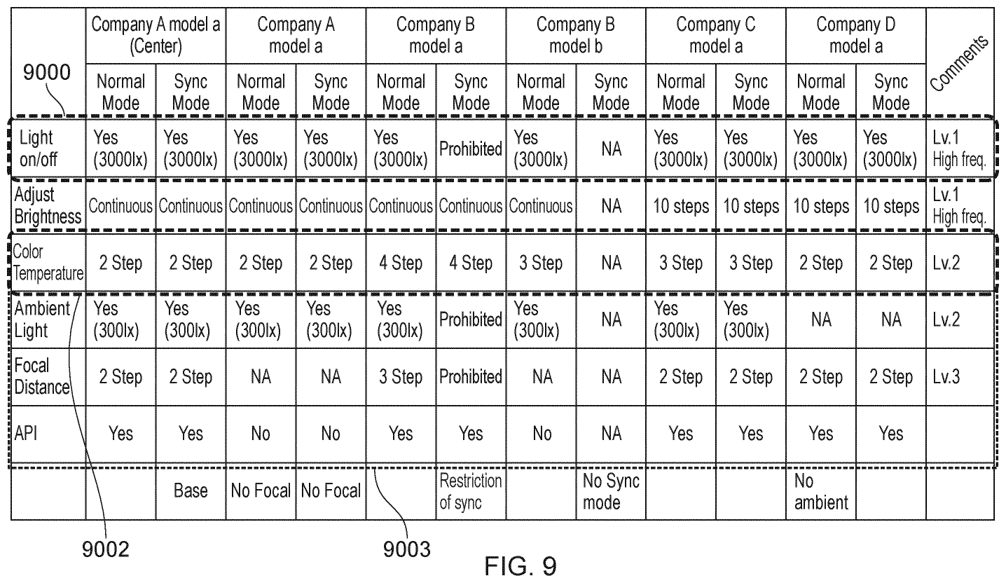

Consider the example of FIG. 9 of the present disclosure. FIG. 9 illustrates an example set of device specifications (being control operations and corresponding available configurations) in accordance with embodiments of the disclosure. The data indicative of frequency of operation is not, itself, shown in this example.

However, for a first user, the Light on/off control operation and the Adjust Brightness control operation are most frequently used when controlling the devices of the medical system. Therefore, these control operations are categorized as a first group 9000 comprising the most frequently used control operations for the user.

Moreover, the Color Temperature control operation is used quite often by the user when controlling the devices of the medical system. Therefore, this Color Temperature control operation forms the second group 9002 of control operations for the user in this example.

However, the control operations Ambient Light, Focal Distance and API are very rarely used by the user. Accordingly, these control operations are categorized as a third group 9003 of control operations for that user.

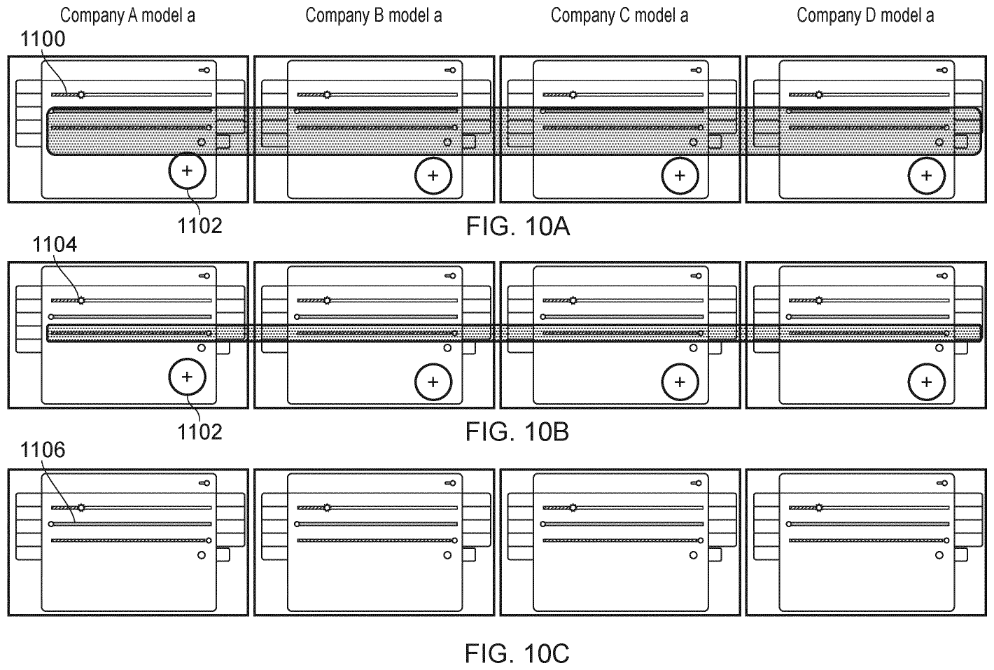

Turning now to FIG. 10A of the present disclosure, an example control display produced in accordance with embodiments of the disclosure is illustrated.

In the example control display (or user interface) which is shown in FIG. 10A, only the first group of control operations 1100 (being those control operations most frequently selected by the user) are shown to the user. Other control operations not forming part of this first group of control operations may be obscured or otherwise not displayed at all. As such, even though the number of common control operations may be large, the user can readily identify and select the desired control operation. This improves the efficiency of operation when controlling devices of the medical system.

However, in some situations, the user may require the use of a control operation which does not form part of the first group of control operations (such as the Color Temperature control operation described with reference to FIG. 9 of the present disclosure). Accordingly, the user may provide an instruction to display the second group of control operations. In examples, this instruction may be provided by interacting with an onscreen element such as button 1102.

When the user provides this instruction, the producing unit 5006 produces a new display signal to cause the display to display both the first and second group of common control operations 1104 to the user. As such, the user will be presented with an updated user interface such as that illustrated in FIG. 10B of the present disclosure.

In some cases, the control operation which the user desires to use may not be present in either the first or the second set of common control operations. Accordingly, the user may provide an instruction to display all of the common control operations. In examples, this instruction may be provided by interacting with an onscreen element such as button 1102. Then, the user will be presented with an updated user interface such as that illustrated in FIG. 10C of the present disclosure. The user may then select the desired control operation from the common set of control operations 1106 which are displayed (being all of the common control operations of the devices of the medical system).

As such, even when the user desires to use a control operation which is not frequently selected, the user can quickly and efficiently access the desired control operation in order to control the devices of the medical system.

### <Method>

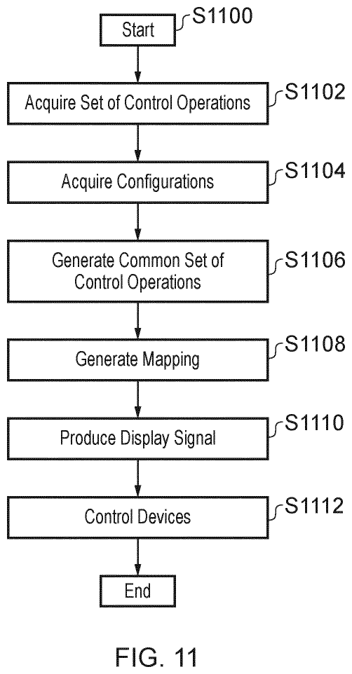

FIG. 11 illustrates a method of controlling operations of a plurality of devices of a medical system in accordance with embodiments of the disclosure. The medical system may be a medical system such as described with reference to the example of FIG. 2 of the present disclosure. Indeed, the medical system may comprise a plurality of different types of devices of a plurality of different specifications, wherein devices of a different type and/or devices of the same type having a different specification have a different set of available control operations.

The method begins at step S1100 and proceeds to step S1102.

In step S1102, the method comprises acquiring information regarding the set of control operations which are available for each device of the plurality of devices of the medical system.

Then, in step S104, the method comprises acquiring information regarding configurations which can be chosen for each type of control operation for each device of the medical system.

Once the information has been acquired, the method proceeds to step S1106.

In step S106, the method comprises generating a common set of control operations for controlling the plurality of devices of the medical system, the common set of control operations comprising at least one type of control operation which can be performed by each device of the plurality of devices of the medical system.

In step S1108, the method comprises generating a mapping between the common set of control operations and the configuration which can be chosen for each type of control operation forming part of the common set of control operations for each device of the plurality of devices of the medical system.

Once the information has been generated, the method proceeds to step S1110.

In step S1110, the method comprises producing a signal to cause an external display device to display the common set of control operations as a single user interface for the control of the plurality of devices of the medical system.

Then, in step S1112, the method comprises controlling one or more of the devices of the medical system using a selection of a control operation from the common set of control operations and the mapping which has been generated.

The method then proceeds to and ends with step S1114.

Of course, the method of controlling operations of a plurality of devices of a medical system is not particularly limited to the example of FIG. 11. A number of modifications to the step of method illustrated in the example of FIG. 11 of the present disclosure may be made depending on the example to which embodiments of the present disclosure are applied. In particular, while the method steps of the example 11 are described in sequence, a number of these steps may, alternatively, be performed in parallel, for example.

### Second Embodiment

Returning now to FIG. 1 of the present disclosure, consider an example situation where a surgeon (or surgical team) located in operation room 1002B performs a surgical operation (such as an endoscopic procedure or the like) on a patient.

At the beginning of the surgical procedure (i.e. during a first stage of the medical procedure before the surgeon begins to use an endoscopic system) the surgeon may require that the surgical lights in the operation room 1002B operate in a high brightness configuration. However, once the surgeon begins to use the endoscopic system, the surgeon may require that the surgical lights in the operation room 1002B are changed to a low brightness setting—such as an ambient light configuration. Indeed, an ambient light control operation or configuration may be a comparatively dark light used in endoscopy surgery.

In some situations, such as where there are a large number of surgical lights in the operations room and/or where there are a number of different specifications of surgical lights in the operation room 1002B, it can be difficult and cumbersome for the surgeon (or surgical team) to control the individual surgical lights such that those lights are changed to operate in a desired configuration (i.e. brightness level in this example). The technical burden increases if the surgeon intermittently requires use of the endoscopic system (i.e. if the configuration of the surgical lights must be changed a number of times). Any difficulty and corresponding delay in the changing of the configuration of the surgical lights can lead to adverse consequences when performing a surgical procedure.

As such, for these reasons (in addition to the reasons discussed in the Background) it is desired that an apparatus, method and computer program product are provided which enables efficient and accurate control of a plurality of devices of a medical system.

### <Apparatus>

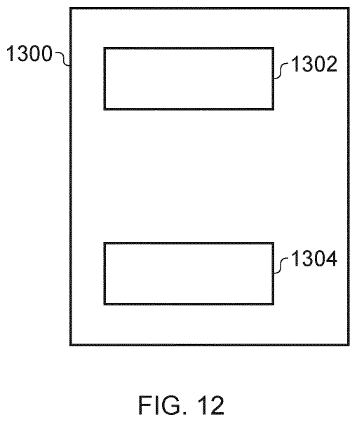

FIG. 12 illustrates an example configuration of a device in accordance with embodiments of the disclosure. The apparatus may be configured for controlling operations of a plurality of devices of a medical system in accordance with embodiments of the disclosure.

The apparatus 1300 comprises an acquiring unit 1302 and a controlling unit 1304.

The acquiring unit 1302 is configured to acquire an activation signal, the activation signal being generated when a user activates a second medical system.

Then, the controlling unit 1304 is configured to control the plurality of devices of the medical system to cause the plurality of devices to operate in a operation mode in accordance with the second medical system which has been activated by the user, wherein the operation mode for each device of the plurality of devices of the medical system is defined by the available control operations for that device.

In this manner, the apparatus 1300 enables control of multiple different types and models of devices with increased accuracy and efficiency.

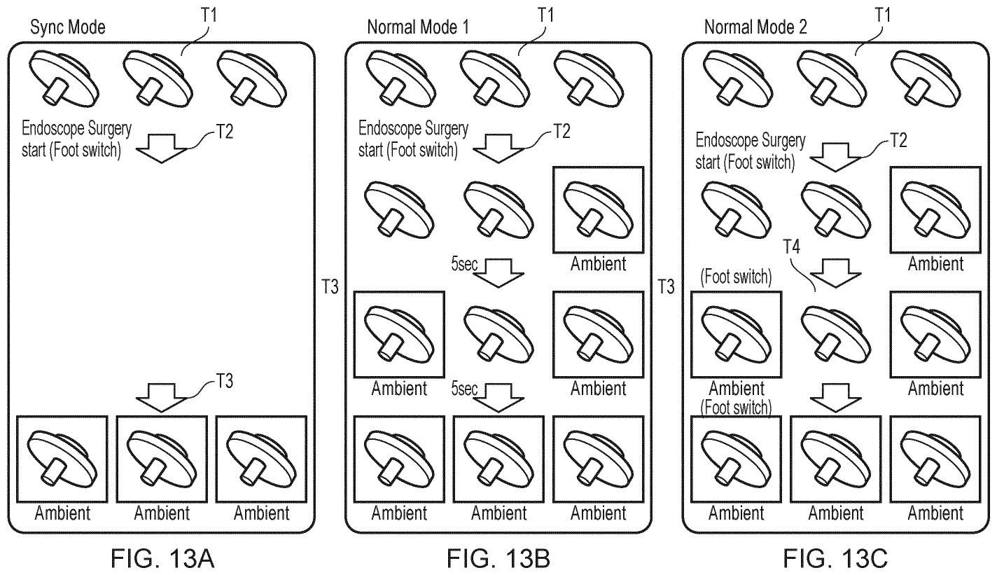

Further details regarding the apparatus 1300 will now be described with reference to FIGS. 13A, 13B and 13C of the present disclosure.

### <Example Timing Chart>

Consider again the situation described with reference to FIG. 1 of the present disclosure where a surgeon is to perform endoscopic surgery on a patient in an operation room such as operation room 1002B.

For minimally invasive surgery such as endoscopic surgery (performed using an endoscopic system) ambient lights as opposed to the normal surgical lights should be used (or, more generally, the surgical lights should operate in an ambient light configuration).

At the start of the surgical procedure, however, the surgical lights may be operating in a bright light mode (i.e. operating as normal surgical lights).

FIG. 13A illustrates an example timing chart in accordance with embodiments of the disclosure. This is a timing chart as may be experienced when performing endoscopic surgery in accordance with embodiments of the disclosure. Time increases from the top FIG. 13A to the bottom of FIG. 13A.

Initially, at time T1 of FIG. 13A, the surgical lights are operating in a bright light mode (i.e. operating as normal surgical lights).

Then, at a time T during a certain stage of the surgical procedure, the surgeon activates an endoscopic surgery system. This indicates the start of endoscopic surgery. The mechanism by which the surgeon activates the endoscopic surgical system is not particularly limited in accordance with embodiments of the disclosure. In some examples, the surgeon may operate a switch (such as a foot switch or the like) in order to activate the surgical system. However, in other examples the endoscopic surgical system may be activated by the surgeon performing any other suitable action (such as issuing a voice instruction or the like).

When the endoscopic surgical system has been activated, the acquiring unit 1302 acquires an activation signal indicating that the endoscopic surgical system has been activated. The activation signal may be transmitted by the endoscopic surgical system itself in some examples.

Once the acquiring unit 1302 has acquired the activation signal, the controlling unit 1304 is configured to control the plurality of devices of the medical system to cause the plurality of devices to operate in a operation mode in accordance with the second medical system which has been activated by the user, wherein the operation mode for each device of the plurality of devices of the medical system is defined by the available control operations for that device.

Specifically, when the activation signal is acquired, the controlling unit is configured to control the surgical lights in order to cause the surgical lights to operate in the ambient light mode. As such, when the surgeon activates the endoscopic system, the surgical lights of operation room 1002B switch to the ambient light configuration under the control of the controlling unit 1304. This occurs at time T3 in the example of FIG. 13A of the present disclosure.

Accordingly, a surgeon (or other user) can efficiently and accurately control the plurality of devices of the medical system (e.g. the surgical lights) in order to change the configuration of those devices.

While, in the example of FIG. 13A, the surgical lights switch to the ambient light configuration, it will be appreciated the present disclosure is not particularly limited in this regard. That is, the configuration to which the devices of the medical system switch will depend on the type of second medical system which has been activated by the surgeon (or other user). For example, if the surgeon activates a second system which requires high brightness (for example) then the controlling unit 1304 will control the configuration of the surgical lights such that they operate in a high brightness configuration. A database of configurations associated with different types of second medical systems may be stored internally or externally to the apparatus 1300. Then, when the activation signal is acquired, the control unit 1304 can select an appropriate configuration in accordance with the type of second medical system which has been active by the user. The database of configuration may, in some examples, be stored in the form of a lookup table or the like. In some examples, the database may include different configurations for different surgeons. In this case, the new configuration (being the configuration to which the lights of the medical system are changed when the second medical system is activated) will depend on the individual preference of the user.

Furthermore, in some examples it may be that one or more of the surgical lights cannot operate in the desired configuration (i.e. the ambient light configuration in this example). That is, as explained with reference to FIG. 4 of the present disclosure, different devices may have different specifications—such that a certain control operation and/or a certain configuration of that control operation may not be available for a given device of the medical system. Accordingly, in some examples the controlling unit 1304 may identify a number of different control operations and/or configurations which are compatible with the second medical system which has been activated for the user. These may be recorded in order of preference in the database, for example. Accordingly, if a surgical light cannot operate in ambient mode (first preference), it may be that the controlling unit 1304 controls the surgical light such that it operates with merely reduced brightness levels (second preference) or is switched off (third preference). As such, even if a surgical light (or other device) of the medical system cannot operate in the preferred configuration, an alternative configuration which is compatible with the second medical which has been activated based on the available control operations for that device.

The example of FIG. 13A of the present disclosure describes a situation where all of the surgical lights of the medical system are changed in synchronization to the preferred operational configuration upon activation of the second medical system. However, the present disclosure is not particularly limited in this regard.

In other examples, the surgeon (or other user) may inform apparatus 1300 that a different mode should be used for the transition of the plurality of devices of the surgical system to the new operational configuration. This may be provided by form of user input prior to the operation, for example. A second example mode (Normal Mode 1) is described with reference to FIG. 13B of the present disclosure.

At time T1 of the example of FIG. 13B, the devices of the medical system (e.g. the surgical lights) are operating in a first configuration (such as a high brightness mode). Then, at a certain stage during the surgical procedure, the surgeon may activate a second medical system which sends an activation signal to apparatus 1300. This may occur at time T of the example of FIG. 13B.

Once the activation signal has been acquired, controlling unit 1304 may control the devices of the medical system to operate in operation mode (or operation configuration) in accordance with the capabilities of the individual devices and the second medical system which has been activated by the user. In this example, the user has activated an endoscopic surgical system and the controlling unit 1304 controls the devices to operate in an ambient light mode or configuration.

However, in contrast to FIG. 13A of the present disclosure, the controlling unit 1304 does not control the devices to operate in the ambient light configuration in synchronization (i.e. with simultaneous control). Rather, the controlling unit 1304 controls the devices of the medical system such that they operate with the ambient light configuration in sequence.

Accordingly, at time T3 of the example of FIG. 13B, the controlling unit 1304 instructs a first one of the devices to operate in the ambient light configuration (this may be a time at (or shortly after) the time at which the activation signal is received). Then, after a predetermined time following T3 (such as five seconds in this example) the controlling unit 1304 causes a second of the devices to operate in the ambient light configuration. A further predetermined delay is then imposed before the next light is changed by the controlling unit 1304 to the new configuration. In fact, this process may continue until all of the devices of the medical system have been changed to operate in the new configuration.

Of course, the present disclosure is not particularly limited to the example of a five second predetermined delay (as indicated in the example of FIG. 13B). Rather, any suitable predetermined time delay between switching of the devices may be used or selected by the user depending on the situation. The predetermined delay may be much longer or much shorter than the five second example provided in FIG. 13B of the present disclosure.

Advantageously, switching the devices in a sequence in accordance with the example of FIG. 13B of the present disclosure ensures that sudden changes in the lighting (caused by simultaneous change of the configuration of all lighting devices upon activation of the second medical system) are avoided. This may provide improved levels of safety and comfort when controlling the plurality of devices of the medical system in accordance with the second embodiment of the disclosure.

Alternatively, in some examples, the controlling unit 1304 may control the switch of the devices of the medical system following the acquisition of the activation signal in sequence, with the next device in the sequence being changed to the new configuration when further input is received from the user.

Consider the example of FIG. 13C of the present disclosure. FIG. 13C illustrates an example timing chart in accordance with embodiments of the disclosure.

Initially, at time T1 of the example of FIG. 13C of the present disclosure, the surgical lights are operating in a first mode of operation (this may be a high brightness configuration, for example). Then, at time T of FIG. 13C, the acquiring unit 1302 of apparatus 1300 acquires an activation signal indicating that the user has activated an endoscopic surgical device. Accordingly, at time T3 of FIG. 13C the controlling unit 1304 of apparatus 1300 controls a first of the surgical lights to change to an ambient light configuration.

At this stage, the other lights of the medical system remain in the high brightness configuration.

Then, at time T4 of FIG. 13C, the user provides a further input which instructs the controlling unit 1304 to change the next light in the sequence into the ambient light configuration. The type of user input is not particularly limited in accordance with embodiments of the disclosure and may, in some examples, include the user pressing a foot switch or the like in order to send a signal to the apparatus 1300.

After the second light has changed to the new configuration, the other lights of the medical system will be changed, in sequence, to the new configuration as the user provides subsequent input instructions to the apparatus 1300.

Advantageously, the method of switching in sequence based on a subsequent user instruction as described with reference to FIG. 13C of the present disclosure provides additional levels of control when switching the devices of the medical system while ensuring that sudden changes in the lighting are avoided.

The sequence (order) in which the surgical lights (or other medical device of the medical system) are switched to the new configuration mode in the examples of FIGS. 13B and 13C of the present disclosure is not particularly limited. In some examples, the sequence of lights may be a predetermined sequence. In other examples, the sequence may be decided at random as further switching instructions are provided. In other examples, the sequence may be indicated by the user as a user preference.

Moreover, while the examples of FIGS. 13B and 13C have been described to a situation where the devices are changed in sequence with a single additional device being changed to the new configuration at a given instance of time, the present disclosure is not so limited. That is, in some examples, a plurality of devices (being still less than the total number of devices of the medical system) may be switched to the new configuration at a given instance of time. For example, two or more devices of the medical system may be changed to the new configuration at time T3 or T4 in certain examples.

In this manner, apparatus 1300 enables efficient and accurate control of the plurality of devices of the medical system.

### <Method>

More generally, a method of controlling operations of a plurality of devices of a medical system is provided.

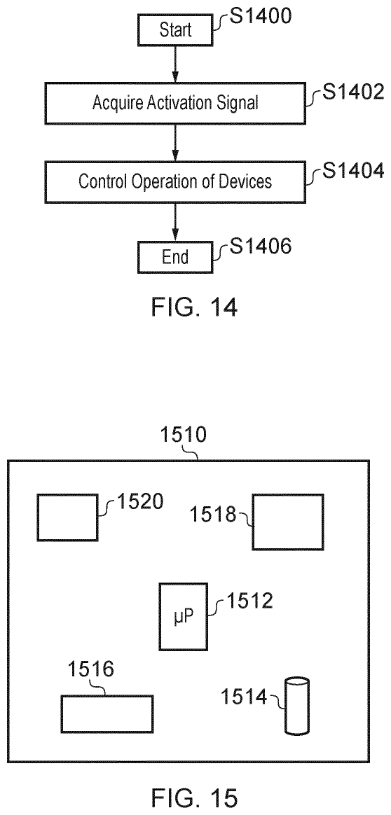

FIG. 14 illustrates a method of controlling operations of a plurality of devices of a medical system in accordance with embodiments of the disclosure. The medical system may be a medical system such as that described with reference to the example of FIG. 2 of the present disclosure. Indeed, the medical system may comprise a plurality of different types of devices of a plurality of different specifications, wherein devices of a different type and/or devices of the same type having a different specification have a different set of available control operations.

The method begins at step S1400 and proceeds to step S1402.

In step S1402, the method comprises acquiring an activation signal, the activation signal being generated when a user activates a second medical system.

Once the activation signal has been acquired, the method proceeds to step S1404.

In step S1404, the method comprises controlling the plurality of devices of the medical system to cause the plurality of devices to operate in an operation mode in accordance with the second medical system which has been activated by the user.

The operation mode for each device of the plurality of devices of the medical system, in this example method, is defined by the available control operations for that device.

The method then proceeds to and ends with step S1406.

Of course, the method of controlling operations of a plurality of devices of a medical system is not particularly limited to the example of FIG. 14. A number of modifications to the step of method illustrated in the example of FIG. 14 of the present disclosure may be made depending on the example to which embodiments of the present disclosure are applied.

### <Example Hardware Configuration>

Referring now to FIG. 15, an apparatus 1510 according to embodiments of the disclosure is shown. This apparatus 1510 may be an example of the hardware configuration of an apparatus such as apparatus 5000 or 1300. Typically, an apparatus 1510 according to embodiments of the disclosure is a computer device such as a personal computer or a terminal connected to a server. Indeed, in embodiments, the apparatus may also be a server. The apparatus 1510 is controlled using a microprocessor or other processing circuitry 1512.

The processing circuitry 1512 may be a microprocessor carrying out computer instructions or may be an Application Specific Integrated Circuit. The computer instructions are stored on storage medium 1514 which may be a magnetically readable medium, optically readable medium or solid state type circuitry. The storage medium 1514 may be integrated into the apparatus 1500 (as shown) or, alternatively, may be separate to the apparatus 1500 and connected thereto using either a wired or wireless connection. The computer instructions may be embodied as computer software that contains computer readable code which, when loaded onto the processor circuitry 1512, configures the processor circuitry 1512 to perform a method according to embodiments of the disclosure.

Now, additionally connected to the processor circuitry 1512, is a user input 1506. The user input 1516 may be a touch screen or maybe a mouse or stylist type input device. The user input 1516 may also be a keyboard or any combination of these devices.

A network connection 1518 is also coupled to the processor circuitry 1512. The network connection 1518 may be a connection to a Local Area Network or a Wide Area Network such as the Internet or a Virtual Private Network or the like. The network connection 1518 may be connected to banking infrastructure allowing the processor circuitry 1512 to communicate with other banking institutions to obtain relevant data or provide relevant data to the institutions. The network connection 1518 may therefore be behind a firewall or some other form of network security. Indeed, network connection 1518 may be used to perform encrypted communication with a connected device.

Additionally coupled to the processing circuitry 1512, is a display device 1520. The display device, although shown integrated into the apparatus 1510, may additionally be separate to the apparatus 1510 and may be a monitor or some kind of device allowing the user to visualise the operation of the system. In addition, the display device 1520 may be a printer or some other device allowing relevant information generated by the apparatus 1510 to be viewed by the user or by a third party.

### <Clauses>

Furthermore, embodiments of the present disclosure may also be arranged in accordance with the following numbered clauses:
1. Apparatus for controlling operations of a plurality of devices of a medical system, the medical system comprising a plurality of different types of devices of a plurality of different specifications, wherein devices of a different type and/or devices of the same type having a different specification have a different set of available control operations, the apparatus comprising circuitry configured to: acquire information regarding the set of control operations which are available for each device of the plurality of devices of the medical system;
acquire information regarding configurations which can be chosen for each type of control operation for each device of the medical system;
generate a common set of control operations for controlling the plurality of devices of the medical system, the common set of control operations comprising at least one type of control operation which can be performed by each device of the plurality of devices of the medical system;
generate a mapping between the common set of control operations and the configuration which can be chosen for each type of control operation forming part of the common set of control operations for each device of the plurality of devices of the medical system;
produce a signal to cause an external display device to display the common set of control operations as a single user interface for the control of the plurality of devices of the medical system; and control one or more of the devices of the medical system using a selection of a control operation from the common set of control operations and the mapping which has been generated.
2. The apparatus according to clause 1, wherein information regarding configurations which can be chosen for each type of control operation include at least one of: values, settings, options and/or scales which can be chosen for each type of control operation.
3. The apparatus according to clause 1 or 2, wherein the single user interface for the control of the plurality of devices of the medical system includes one or more selectable elements for each of the common set of control operations for each device.
4. The apparatus according to any preceding clause, wherein the single user interface for the control of the plurality of devices of the medical system includes one or more selectable elements for each of the common set of control operations for synchronised control of each device.
5. The apparatus according to any preceding clause, wherein the apparatus is configured to restrict one or more of the common set of control operations for one or more of the plurality of devices of the medical system.
6. The apparatus according to clause 5, wherein restricting a control operation includes limiting the configurations which can be chosen for that control operation for a certain device.
7. The apparatus according to clause 5, wherein restricting a control operation includes prohibiting the control operation for being activated for a certain device in one or more situations.
8. The apparatus according to clause 5, wherein the apparatus is further configured to produce a display signal to adapt the signal user interface for the control of the plurality of devices of the medical system to restrict one or more of the common set of control operations for one or more of the plurality of devices of the medical system.
9. The apparatus according to any preceding clause, wherein the apparatus is further configured to categorize the one or more control operations of the common set of control operations in accordance with a frequency of selection.
10. The apparatus according to clause 9, wherein the control operations of the common set of control operations having a frequency of selection above a first predetermined threshold value are categorized as a first group of control operations; and wherein the apparatus is configured to produce a display signal to cause the external display device to display the first group of control operations as a single user interface for the control of the plurality of devices of the medical system.
11. The apparatus according to clause 10, wherein the apparatus is configured to produce a display signal to cause the external display device to display the first group of control operations and a second group of control operations as a single user interface for the control of the plurality of devices of the medical system in accordance with a selection of a user; wherein the second group of control operations are control operations of the common set of control operations having a frequency of selection below the first predetermined threshold value and above a second predetermined threshold value.
12. The apparatus according to any preceding clause, the plurality of devices of the medical system being surgical lighting devices of a plurality of different types and/or a plurality of different specifications.
13. An apparatus for controlling operations of a plurality of devices of a medical system, the medical system comprising a plurality of different types of devices of a plurality of different specifications, wherein devices of a different type and/or devices of the same type having a different specification have a different set of available control operations, the apparatus comprising circuitry configured to:
acquire an activation signal, the activation signal being generated when a user activates a second medical system; and
control the plurality of devices of the medical system to cause the plurality of devices to operate in an operation mode in accordance with the second medical system which has been activated by the user;
wherein the operation mode for each device of the plurality of devices of the medical system is defined by the available control operations for that device.
14. The apparatus according to clause 13, the plurality of devices of the medical system being surgical lighting devices of a plurality of different types and/or a plurality of different configurations and the second medical system being an endoscopic surgical system.
15. The apparatus according to clause 14, wherein the operation mode of the plurality of surgical lighting devices when the endoscopic surgical system has been activated by the user is an ambient lighting mode.
16. The apparatus according to clause 15, wherein the apparatus controls the surgical lighting devices to cause the plurality of surgical lighting devices to operate in the ambient lighting mode in synchronization.
17. The apparatus according to clause 16, wherein the apparatus controls the surgical lighting device to cause the plurality of surgical lighting devices to operate in the ambient lighting mode in sequence with a predetermined time delay between each surgical lighting device.
18. The apparatus according to clause 16, wherein the apparatus controls the surgical lighting devices to cause the plurality of surgical lighting devices to operate in the ambient lighting mode in sequence, wherein each surgical lighting device in the sequence is controlled in turn in accordance with additional user input acquired from a user input device.
19. Method of controlling operations of a plurality of devices of a medical system, the medical system comprising a plurality of different types of devices of a plurality of different specifications, wherein devices of a different type and/or devices of the same type having a different specification have a different set of available control operations, the method comprising the steps of:
acquiring information regarding the set of control operations which are available for each device of the plurality of devices of the medical system;
acquiring information regarding configurations which can be chosen for each type of control operation for each device of the medical system;
generating a common set of control operations for controlling the plurality of devices of the medical system, the common set of control operations comprising at least one type of control operation which can be performed by each device of the plurality of devices of the medical system;
generating a mapping between the common set of control operations and the configuration which can be chosen for each type of control operation forming part of the common set of control operations for each device of the plurality of devices of the medical system;
producing a signal to cause an external display device to display the common set of control operations as a single user interface for the control of the plurality of devices of the medical system; and
controlling one or more of the devices of the medical system using a selection of a control operation from the common set of control operations and the mapping which has been generated.
20. Method of controlling operations of a plurality of devices of a medical system, the medical system comprising a plurality of different types of devices of a plurality of different specifications, wherein devices of a different type and/or devices of the same type having a different specification have a different set of available control operations, the method comprising the steps of:
acquiring an activation signal, the activation signal being generated when a user activates a second medical system; and
controlling the plurality of devices of the medical system to cause the plurality of devices to operate in an operation mode in accordance with the second medical system which has been activated by the user;
wherein the operation mode for each device of the plurality of devices of the medical system is defined by the available control operations for that device.
21. Computer program product comprising instructions which, when the instructions are implemented by a computer, cause the computer to perform a method of controlling operations of a plurality of devices of a medical system according to clause 19 or clause 20.

While certain embodiments of the disclosure have been described with reference to the medical facility illustrated with reference to FIG. 1 of the present disclosure, it will be appreciated that the present disclosure is not particularly limited in this regard. That is, embodiments of the present disclosure may, alternatively, be applied to any medical system including any number of a plurality of devices.

Indeed, while certain embodiments of the disclosure have been described specifically with reference to a plurality of surgical lights as the plurality of devices of the medical system, it will be appreciated that other medical devices (and not only surgical lights) may be controlled in accordance with the embodiments of the disclosure. These devices may include any of the devices as described with reference to FIG. 2 of the present disclosure, for example.

However, in all these examples, the advantageous technical effect of improved accuracy and efficiency of operation of the devices of the medical system will be achieved by apparatus 5000 and/or apparatus 1300 of the present disclosure.

Obviously, numerous modifications and variations of the present disclosure are possible in light of the above teachings. It is therefore to be understood that within the scope of the appended claims, the disclosure may be practiced otherwise than as specifically described herein.

In so far as embodiments of the disclosure have been described as being implemented, at least in part, by software-controlled data processing apparatus, it will be appreciated that a non-transitory machine-readable medium carrying such software, such as an optical disk, a magnetic disk, semiconductor memory or the like, is also considered to represent an embodiment of the present disclosure.

It will be appreciated that the above description for clarity has described embodiments with reference to different functional units, circuitry and/or processors. However, it will be apparent that any suitable distribution of functionality between different functional units, circuitry and/or processors may be used without detracting from the embodiments.

Described embodiments may be implemented in any suitable form including hardware, software, firmware or any combination of these. Described embodiments may optionally be implemented at least partly as computer software running on one or more data processors and/or digital signal processors. The elements and components of any embodiment may be physically, functionally and logically implemented in any suitable way. Indeed the functionality may be implemented in a single unit, in a plurality of units or as part of other functional units. As such, the disclosed embodiments may be implemented in a single unit or may be physically and functionally distributed between different units, circuitry and/or processors.

Although the present disclosure has been described in connection with some embodiments, it is not intended to be limited to the specific form set forth herein. Additionally, although a feature may appear to be described in connection with particular embodiments, one skilled in the art would recognize that various features of the described embodiments may be combined in any manner suitable to implement the technique.

1. Apparatus for controlling operations of a plurality of devices of a medical system, the medical system comprising a plurality of different types of devices of a plurality of different specifications, wherein devices of a different type and/or devices of the same type having a different specification have a different set of available control operations, the apparatus comprising circuitry configured to:
acquire information regarding the set of control operations which are available for each device of the plurality of devices of the medical system;
acquire information regarding configurations which can be chosen for each type of control operation for each device of the medical system;
generate a common set of control operations for controlling the plurality of devices of the medical system, the common set of control operations comprising at least one type of control operation which can be performed by each device of the plurality of devices of the medical system;
generate a mapping between the common set of control operations and the configuration which can be chosen for each type of control operation forming part of the common set of control operations for each device of the plurality of devices of the medical system;
produce a signal to cause an external display device to display the common set of control operations as a single user interface for the control of the plurality of devices of the medical system; and
control one or more of the devices of the medical system using a selection of a control operation from the common set of control operations and the mapping which has been generated.

2. The apparatus according to claim 1, wherein information regarding configurations which can be chosen for each type of control operation include at least one of: values, settings, options and/or scales which can be chosen for each type of control operation.

3. The apparatus according to claim 1, wherein the single user interface for the control of the plurality of devices of the medical system includes one or more selectable elements for each of the common set of control operations for each device.

4. The apparatus according to claim 1, wherein the single user interface for the control of the plurality of devices of the medical system includes one or more selectable elements for each of the common set of control operations for synchronised control of each device.

5. The apparatus according to claim 4, wherein the apparatus is configured to restrict one or more of the common set of control operations for one or more of the plurality of devices of the medical system.

6. The apparatus according to claim 5, wherein restricting a control operation includes limiting the configurations which can be chosen for that control operation for a certain device.

7. The apparatus according to claim 5, wherein restricting a control operation includes prohibiting the control operation for being activated for a certain device in one or more situations.

8. The apparatus according to claim 5, wherein the apparatus is further configured to produce a display signal to adapt the signal user interface for the control of the plurality of devices of the medical system to restrict one or more of the common set of control operations for one or more of the plurality of devices of the medical system.

9. The apparatus according to claim 1, wherein the apparatus is further configured to categorize the one or more control operations of the common set of control operations in accordance with a frequency of selection.

10. The apparatus according to claim 9, wherein the control operations of the common set of control operations having a frequency of selection above a first predetermined threshold value are categorized as a first group of control operations; and wherein the apparatus is configured to produce a display signal to cause the external display device to display the first group of control operations as a single user interface for the control of the plurality of devices of the medical system.

11. The apparatus according to claim 10, wherein the apparatus is configured to produce a display signal to cause the external display device to display the first group of control operations and a second group of control operations as a single user interface for the control of the plurality of devices of the medical system in accordance with a selection of a user; wherein the second group of control operations are control operations of the common set of control operations having a frequency of selection below the first predetermined threshold value and above a second predetermined threshold value.

12. The apparatus according to claim 1, the plurality of devices of the medical system being surgical lighting devices of a plurality of different types and/or a plurality of different specifications.

13. An apparatus for controlling operations of a plurality of devices of a medical system, the medical system comprising a plurality of different types of devices of a plurality of different specifications, wherein devices of a different type and/or devices of the same type having a different specification have a different set of available control operations, the apparatus comprising circuitry configured to:
acquire an activation signal, the activation signal being generated when a user activates a second medical system; and
control the plurality of devices of the medical system to cause the plurality of devices to operate in an operation mode in accordance with the second medical system which has been activated by the user;
wherein the operation mode for each device of the plurality of devices of the medical system is defined by the available control operations for that device.

14. The apparatus according to claim 13, the plurality of devices of the medical system being surgical lighting devices of a plurality of different types and/or a plurality of different configurations and the second medical system being an endoscopic surgical system.

15. The apparatus according to claim 14, wherein the operation mode of the plurality of surgical lighting devices when the endoscopic surgical system has been activated by the user is an ambient lighting mode.

16. The apparatus according to claim 15, wherein the apparatus controls the surgical lighting devices to cause the plurality of surgical lighting devices to operate in the ambient lighting mode in synchronization.

17. The apparatus according to claim 16, wherein the apparatus controls the surgical lighting device to cause the plurality of surgical lighting devices to operate in the ambient lighting mode in sequence with a predetermined time delay between each surgical lighting device.

18. The apparatus according to claim 16, wherein the apparatus controls the surgical lighting devices to cause the plurality of surgical lighting devices to operate in the ambient lighting mode in sequence, wherein each surgical lighting device in the sequence is controlled in turn in accordance with additional user input acquired from a user input device.

19. Method of controlling operations of a plurality of devices of a medical system, the medical system comprising a plurality of different types of devices of a plurality of different specifications, wherein devices of a different type and/or devices of the same type having a different specification have a different set of available control operations, the method comprising the steps of:
acquiring information regarding the set of control operations which are available for each device of the plurality of devices of the medical system;
acquiring information regarding configurations which can be chosen for each type of control operation for each device of the medical system;
generating a common set of control operations for controlling the plurality of devices of the medical system, the common set of control operations comprising at least one type of control operation which can be performed by each device of the plurality of devices of the medical system;
generating a mapping between the common set of control operations and the configuration which can be chosen for each type of control operation forming part of the common set of control operations for each device of the plurality of devices of the medical system;
producing a signal to cause an external display device to display the common set of control operations as a single user interface for the control of the plurality of devices of the medical system; and
controlling one or more of the devices of the medical system using a selection of a control operation from the common set of control operations and the mapping which has been generated.

20. Method of controlling operations of a plurality of devices of a medical system, the medical system comprising a plurality of different types of devices of a plurality of different specifications, wherein devices of a different type and/or devices of the same type having a different specification have a different set of available control operations, the method comprising the steps of:
acquiring an activation signal, the activation signal being generated when a user activates a second medical system; and
controlling the plurality of devices of the medical system to cause the plurality of devices to operate in an operation mode in accordance with the second medical system which has been activated by the user;
wherein the operation mode for each device of the plurality of devices of the medical system is defined by the available control operations for that device.

21. One or more non-transitory computer-readable media comprising instructions which, when the instructions are implemented by a computer, cause the computer to perform a method of controlling operations of a plurality of devices of a medical system according to claim 19.
